(19) United States
(12) Patent Application Publication
(10) Pub. No.: US 2012/0059029 A1
(43) Pub. Date: Mar. 8, 2012

(54) SELECTIVE INTEGRIN INHIBITORS

(75) Inventors: Anthony Pepio, Carlsbad, CA (US); Gordon Francis, San Francisco, CA (US); Theodore A. Yednock, Forest Knolls, CA (US); Alissa Chackerian, Sunnyvale, CA (US); Brian Todd Wipke, Lexington, MA (US)

(73) Assignee: Elan Pharmaceuticals, Inc., South San Francisco, CA (US)

(21) Appl. No.: 13/098,538

(22) Filed: May 2, 2011

Related U.S. Application Data
(60) Provisional application No. 61/330,122, filed on Apr. 30, 2010.
(60) Provisional application No. 61/392,288, filed on Oct. 12, 2010.
(60) Provisional application No. 61/516,845, filed on Apr. 8, 2011.

(51) Int. Cl. A61K 31/437; A61P 35/00; A61P 29/00; A61P 37/02

(52) U.S. Cl. 514/303

(57) ABSTRACT

This invention provides methods for selectively antagonizing the α4β1 integrin heterodimer in a subject in need thereof, and preferably in a human subject. The methods of the invention utilize conjugates comprising two or more α4β1 small molecule antagonists covalently attached to a biocompatible polymer. These methods of selectively inhibiting α4β1 may be used modulate both normal and pathological biological processes mediated at least in part through α4β1 activity.

### CROSS REFERENCE TO RELATED APPLICATIONS

This application claims priority to U.S. Provisional Application Ser. No. 61/330,122, filed Apr. 30, 2010, U.S. Provisional Application Ser. No. 61/392,288, filed Oct. 12, 2010, U.S. Provisional Application Ser. No. 61/516,845, filed Apr. 8, 2011, each of which is incorporated herein by reference in its entirety.

### FIELD OF THE INVENTION

This invention relates to selective inhibitors of integrins, and more specifically to selective inhibitors of α4β1.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Integrin receptors are transmembrane, non-covalently linked heterodimers consisting of one α-chain and one β-chain. In addition to performing a structural adhesive function, integrin receptors transmit extracellular signals across the plasma membrane. The integrin receptor α4β1 (also called VLA-4) mediates cell adhesion by binding with either of two protein ligands: vascular cell adhesion molecule-1 (VCAM-1) (Osborn, L. et al., Cell, 1989, 59, 1203), or the alternatively spliced fibronectin variant containing the type III connecting segment (CS-1) (Wayner, E. A. et al., Cell Biol., 1989, 109, 1321). In contrast to the prototypical integrin receptors that recognize the Arg-Gly-Asp (RGD) tripeptide sequence in their respective ligands, α4β1 recognizes Gln-Ile-Asp-Ser (QIDS) in VCAM-1 and Ile-Leu-Asp-Val (ILDV) in fibronectin. Although these sequences share a conserved Asp residue with RGD, they are otherwise unrelated.

The α4β1 integrin receptor is expressed at high levels on mast cells, mononuclear leukocytes, eosinophils, macrophages, and basophils (Adams, S. P. et al., Ann. Rep. Med. Chem., 1999, 34, 179). The binding of α4β1 to cytokine-induced VCAM-1 at sites of inflammation results in leukocyte/endothelium adhesion followed by extravasation into the inflamed tissue (Chuluyan, H. E. et al., Springer Semin. Immunopathol., 1995, 16, 391). The α4β1 receptor interaction with VCAM-1 also exerts an important effect in stem cell adhesion to bone marrow stromal cells (Simmons, P. J. et al., Blood, 1992, 80, 388).

Cellular adhesion and migration mediated through the β1 integrins are critical components of cellular recruitment processes. The integrin α4β1 provides a key costimulatory signal supporting cell activation leading to growth factor and cytokine production and mediator release. Through recognition of the extracellular matrix, α4β1 increases the survival of activated cells by inhibiting apoptosis (Yoshikawa, H. et al., J. Immunol., 1996, 156, 1832).

As integrins can have various α and β components, therapeutics that hit one or the other of the α4β1 also hit other integrin combinations. For example, therapeutics that selectively bind to the α4 integrin subunit will bind to and modulate both the α4β1 and αβ7 heterodimers. For many disease states, it is preferable to have a therapeutic that selectively binds the heterodimer combination, as this results in a more tailored treatment of the disease state without the potential for adverse events stemming from the binding of other heterodimers.

There is thus a need for compositions and methods of treatment using compounds that selectively bind to α4β1 integrin and that have little effect on signaling through α4β7 and/or processes that utilize α4β7-mediated related pathways. The present invention addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

This invention provides methods for selectively antagonizing the α4β1 integrin heterodimer in a subject in need thereof, and preferably in a human subject. The methods of the invention utilize conjugates comprising two or more α4β1 small molecule antagonists covalently attached to a biocompatible polymer. These methods of selectively inhibiting α4β1 may be used to modulate both normal and/or pathological biological processes mediated at least in part through α4β1 activity.

The present invention is thus directed to a method for the treatment of diseases, disorders or injury by selective inhibition of the α4β1 heterodimer. Such diseases include, but are not limited to, inflammatory disease, autoimmune disease and cell-proliferative disorders. A therapeutically effective amount of such conjugates for use in the present invention may be provided to a subject through various administration routes, and optionally can be provided with pharmaceutically acceptable carriers.

In a specific aspect, the present invention is directed to methods of selectively inhibiting α4β1 by use of conjugates comprising compounds such as those shown in Formula (I):

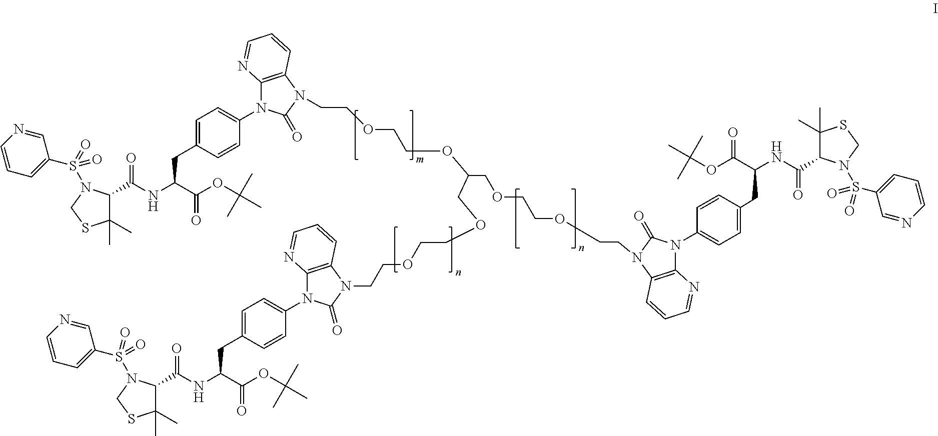

and conjugates comprising related pharmaceutically acceptable salts, racemic mixtures, diastereomers and enantiomers of such compounds. The number of alkylene oxide CH₂—CH₂—O repeating units depicted in Formula (I) vary to provide an average molecular weight of the total amount of polymer arising from single or multiple polymer moieties in the conjugates of between about 100 to 100,000; preferably from about 20,000 to 60,000, also expressed as about 20 to about 60 kiloDaltons (kDa); more preferably from about 30,000 to about 50,000 (or about 30 to about 50 kDa). It is apparent to those skilled in the art that synthetic organic polymers of this type will be polydisperse. Polydispersity refers to the fact that polymer molecules of the lengths contemplated herein, even ones of the same type, come in different sizes (chain lengths, for linear or multi-armed polymers). Therefore average molecular weight will depend on the method of averaging. The polydispersity index (PDI), a common measure of the variability of molecular weights is the ratio of the weight average molecular weight to the number average molecular weight. The PDI is a measure of the distribution of molecular mass in a given polymer sample. The PDI calculated is the weight average molecular weight divided by the number average molecular weight. It indicates the distribution of individual molecular masses in a batch of polymers. For the conjugates of the invention the PDI has a value greater than 1, but as the polymer chains approach uniform chain length, the PDI approaches unity (1). It indicates the distribution of individual molecular weights in a batch of polymers. The number average molecular weight is a way of determining the molecular weight of a polymer. The number average molecular weight is the common average of the molecular weights of the individual polymers. It is determined by measuring the molecular weight of n polymer molecules, summing the weights, and dividing by n. The number average molecular weight of a polymer can be determined by osmometry, end-group titration, and colligative properties.

The weight average molecular weight can be determined by light scattering, small angle neutron scattering (SANS), X-ray scattering, and sedimentation velocity. The ratio of the weight average to the number average is called the polydispersity index. A theoretical sample of polymer having no dispersity would have a polydispersity index of 1. Preferred range of polydispersity index for the present invention is from about 1.10 to about 1.05. More preferred is a range from about 1.05 to the upper limit of commercially feasible synthesis, which to date is about 1.02. The conjugate of formula I is also described in US Appln. No. 20060013799, which is incorporated by reference in its entirety

Other conjugate compositions that are also envisioned for use in the methods of the invention include specific α4β1-selective conjugates comprising two or more α4β1-specific small molecule antagonists and a biocompatible polymer, such as those that are individually described in US Appln. No. 20060013799.

In one aspect, the invention provides specific methods for selectively inhibiting α4β1-mediated leukocyte trafficking. It is a feature of the invention that these methods provide treatments for diseases, disorders and injury by specifically inhibiting α4β1-mediated leukocyte trafficking, including into the central nervous system (CNS), while preserving α4β7-mediated function and trafficking.

In one specific aspect of the invention, the disorders treated using the method of the invention include, but are not limited to, autoimmune diseases such as multiple sclerosis, rheumatoid arthritis, asthma, psoriasis, inflammatory bowel disease and the like.

In another specific aspect of the invention, the disorders treated using the methods of the invention include, but are not limited to, inflammatory diseases such as restenosis and atherosclerosis.

In other specific aspects, the disorders treated using the methods of the invention include, but are not limited to, cell proliferative disorders such as hematopoietic neoplasms, which are abnormal growth involving cells of the hematopoietic lineage, and in particular B cell malignancies.

In yet another specific aspect of the invention, the methods of the invention are use to promote immune suppression, e.g., for the prevention transplant rejection or graft versus host disease (GVHD).

In still other specific aspects, the invention provides methods for inducing neuroprotection following injury, such as spinal cord injury.

In one aspect, the invention provides use of a therapeutically effective dose of Formula (I) to inhibit α4β1-mediated leukocyte trafficking into the CNS while substantially preserving α4β7-mediated trafficking. In one aspect, the dose of Formula (I) is typically from about 0.01 mg/kg to about 10 mg/kg, and preferably from about 0.2 mg/kg to about 1.0 mg/kg. In another aspect, the dose is from about 1.0 mg/kg to about 2.0 mg/kg. In a specific aspect the dose is about 0.5 mg/kg. The dose is preferably administered weekly or monthly.

Another aspect of the invention provides use of a therapeutically effective dose of a conjugate comprising two or more α4β1 small molecule antagonists covalently attached to a biocompatible polymer, wherein the conjugate has a 20 to 100 fold greater potency towards α4β1 than α4β7, in the modulation of a biological process mediated at least in part through α4β1 activity. Preferably, the conjugate has a 30 to 80 fold greater potency towards α4β1 than α4β7, such as the compound of Formula (I).

In other aspects, the invention provides the use of Formula (I) for the treatment of a patient in need thereof comprising administering to the patient a weekly or monthly dose of Formula (I) of from about 0.5 mg/kg to about 2.0 mg/kg.

In yet other aspects, the invention provides the use of Formula (I) for the treatment of a patient with an autoimmune disease comprising administering to the patient a weekly or monthly dose of Formula (I) of from about 0.5 mg/kg to about 2.0 mg/kg.

These and other aspects and uses of the invention will be described in more detail in the written description and as illustrated in the accompanying Figures.

### BRIEF DESCRIPTION OF THE FIGURES

FIG. 1 is a line graph illustrating the competitive binding of the compound of Formula (I) compared to a multivalent α4β1 ligand.

FIG. 2 is a line graph showing the ability of the compound of Formula (I) to disrupt α4β1-Dependent Jurkat™ Adhesion to rs-hu-VCAM-1.

FIG. 3 is a line graph showing the ability Formula (I) to inhibit the α4β1-dependent adhesion of human lymphocytic cells (Jurkat™ cell line) to fibronectin (FN).

FIG. 4 is a line graph showing the ability of Formula (I) to inhibit the binding of human serum FN to activated α4β1 on Jurkat™ cells.

FIG. 5 is a line graph showing Formula (I) plasma concentrations in human subjects receiving various doses of the conjugate.

FIG. 6 is a line graph showing saturation levels of α4β1 and α4β7 on lymphocytes following Formula (I) administration of various doses to human subjects.

FIG. 7 is a bar graph showing average receptor down regulation following a 0.5 mg/kg dose of Formula (I) to a cohort of human subjects.

FIG. 8 is a bar graph showing individual receptor down regulation following a 0.5 mg/kg dose of Formula (I) to human subjects.

FIG. 9 is a bar graph showing patient lymphocyte counts at different dosages of Formula (I).

FIG. 10 is a line graph illustrating the relationship between plasma concentrations of Formula (I) and relative lymphocyte count in patients.

FIG. 11 is a set of line graphs illustrating the receptor occupancy of α4β1 and α4β7 at various doses of Formula (I).

FIG. 12 is a line graph illustrating the receptor saturation of α4β1 and α4β7 at a 0.5 mg/kg dosage of Formula (I).

FIG. 13 is a line graph illustrating the normalized receptor expression of α4β1 and α4β7 at a 0.5 mg/kg dosage of Formula (I).

FIG. 14 is a line graph illustrating the receptor saturation of α4β1 and α4β7 at a 1.0 mg/kg dosage of Formula (I).

FIGS. 15A and 15B are two line graphs illustrating inhibition levels of α4β1 and α4β7 for both the compound of Formula (I) and ELN 476063.

FIGS. 16A through 16F are a series of line graphs illustrating that Formula (I) differentially down-regulates soluble VCAM-1 (sVCAM-1) and soluble MadCAM-1 (sMadCAM-1).

FIGS. 17A and 17B are bar charts correlating plasma compound level with plasma sVCAM-1 and sMAdCAM-1.

FIGS. 18A and 18B are line graphs illustrating the differential regulation of sVCAM-1 and sMAdCAM-1 by Formula (I) and ELN476063.

FIG. 19 are line graphs plotting radioactivity measurements of the compound of Formula (I) (squares) and ELN 476063 (circles) in various murine tissues represented by squares represented by circles.

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The term “administering” as used herein includes the treatment of the various disorders described with compositions comprising the conjugates of the invention The term “administering” also includes single doses as well as dosage regimes in which multiple doses are provided at different times during the course of therapy. Either the single dose or the multiple doses of a dosage regime can be administered in combination forms with other active agents.

The term “α4β1-selective” as used herein refers to a compound that preferentially binds to and modulates the α4β1 heterodimer over other integrin heterodimers, and in particular over α4β7. The α4β1-selective molecules for use in the methods of the invention are characterized by at least a 20 to 100 fold greater potency towards α4β1 than α4β7, and more particularly a 30 to 80 fold greater potency towards α4β1 over α4β7.

The term “autoimmune disease” as used herein refers to diseases and disorders that are characterized by the production of antibodies that react with host tissues or immune effector cells that are autoreactive to endogenous peptides.

The term “cell proliferative disorders” as used herein refers to any disease or disorder associated with abnormal cell growth or cellular activity, including but not limited to cell dedifferentiation or unregulated cell division.

The term “immune suppression” as used herein refers to suppression of the immune response, e.g., in order to prevent the rejection of grafts or transplants or to control autoimmune diseases.

The term “inflammatory disorders” as used herein refers to any disease or disorders that are characterized by pathological processes characterized by injury or destruction of tissues caused by a variety of cytologic and chemical reactions.

The term “subject” as used herein, refers to an animal, preferably a mammal, most preferably a human.

The term “therapeutically effective amount” as used herein, means an amount of the conjugate of the present invention to elicit the desired biological or medical response.

The terms “treat,” “treatment,” “treating,” and the like, refer to obtaining a desired pharmacologic and/or physiologic effect. “Treatment,” as used herein, covers any treatment to achieve a desired effect in a mammal, particularly in a human, and includes: (a) preventing the disease or physiologic effect from occurring in a subject which may be predisposed to the disease or physiologic effect but has not yet been diagnosed as having it; (b) inhibiting the disease or physiologic effect, i.e., arresting its development; and (c) relieving the disease or physiologic effect, e.g., causing regression, e.g., to completely or partially remove symptoms of the disease or physiologic effect. The effect of the treatment may be prophylactic in terms of completely or partially preventing a disease, physiologic effect or symptom thereof, or in maintaining a physiologic state that prevents an adverse reaction such as tissue rejection. The effect may also be therapeutic in terms of a partial or complete cure for response to a disease or physiologic effect and/or adverse affect attributable to the disease or physiologic effect.

### DETAILED DESCRIPTION OF THE INVENTION

The practice of the techniques described herein may employ, unless otherwise indicated, conventional techniques and descriptions of organic chemistry, pharmaceutical chemistry, polymer technology, cell biology, biochemistry, which are within the skill of those who practice in the art. Specific illustrations of suitable techniques, including techniques for the preparation of pharmaceutical preparations comprising the compositions of the invention, can be had by reference to the description and examples herein. However, other equivalent conventional procedures can, of course, also be used. Such conventional techniques and descriptions can be found in standard laboratory manuals such as Lehninger, Principles of Biochemistry 3rd Ed., W. H. Freeman Pub., New York, N.Y.; and Berg et al. (2002) Biochemistry, 5th Ed., W.H. Freeman Pub., New York, N.Y. Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc. Descriptions of pharmaceutically acceptable carriers may be found in The Handbook of Pharmaceutical Excipients, published by the American Pharmaceutical Association and the Pharmaceutical Society of Great Britain. Each of these publications is incorporated by reference in their entirety for use in the invention.

Note that as used herein and in the appended claims, the singular forms “a,” “an,” and “the” include plural referents unless the context clearly dictates otherwise. Thus, for example, reference to “an excipient” refers to one or more excipients, and reference to “the dosage regime” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, well-known features and procedures well known to those skilled in the art have not been described in order to avoid obscuring the invention.

The methods of the invention provide treatment of α4β1-mediated disorders in a subject in need thereof comprising administering to the subject a therapeutically effective amount of an α4β1-selective inhibitor such as specific α4β1-selective antagonists disclosed in US Appln. No. 20060013799. A preferred aspect of the invention a method for the treatment of α4β1-mediated disorders in a subject in need thereof comprising administering to the subject a therapeutically effective amount of the compound of Formula (I).

In specific aspects, the therapeutically effective amount of the compound is from about 0.01 mg/kg/day to about 30 mg/kg/day.

The compositions of the present invention comprising the α4β1-selective conjugates may be administered by any conventional route of administration including, but not limited to oral, nasal, pulmonary, sublingual, ocular, transdermal, rectal, vaginal and parenteral (i.e. subcutaneous, intramuscular, intradermal, intravenous etc.). Subcutaneous, intramuscular or intradermal routes of administration are preferred.

In preparing a composition of the present invention in liquid dosage form for oral, topical and parenteral administration, any of the usual pharmaceutical media or excipients may be employed. Thus, for liquid dosage forms, such as suspensions (i.e. colloids, emulsions and dispersions) and solutions, suitable carriers and additives include but are not limited to pharmaceutically acceptable wetting agents, dispersants, flocculation agents, thickeners, pH control agents (i.e. buffers), osmotic agents, coloring agents, flavors, fragrances, preservatives (i.e. to control microbial growth, etc.) and a liquid vehicle may be employed. In the case of parenteral formulations sterile, non-pyrogenic liquid vehicles are preferred. Not all of the components listed above will be required for each liquid dosage form.

In solid oral preparations such as, for example, dry powders for reconstitution or inhalation, granules, capsules, caplets, gelcaps, pills and tablets (each including immediate release, timed release and sustained release formulations), suitable carriers and additives include but are not limited to diluents, granulating agents, lubricants, binders, glidants, disintegrating agents and the like. Because of their ease of administration, tablets and capsules represent the most advantageous oral dosage unit form, in which case solid pharmaceutical carriers are obviously employed. If desired, tablets may be sugar coated, gelatin coated, film coated or enteric coated by standard techniques.

The compositions comprising the α4β1-selective conjugates will contain, per dosage unit, e.g., tablet, capsule, powder, injection, teaspoonful and the like, an amount of the conjugate necessary to deliver an effective dose as described herein.

The pharmaceutical compositions herein will contain, per unit dosage unit, e.g., tablet, capsule, powder, sterile solution or suspension for injection, suppository, and the like, of from about 0.01 mg/kg to about 300 mg/kg (preferably from about 0.1 mg/kg to about 10 mg/kg; and, more preferably, from about 0.5 mg/kg to about 2.0 mg/kg) and may be given at a dosage that provides above 80% α4β1 receptor saturation over a given time period. Dosage will depend on the time interval between administrations.

Preferably, the method for the treatment of α4β1 integrin-mediated disorders described in the present invention using the α4β1-conjugates will be administered on a weekly or monthly basis, and will use a dosage form containing a pharmaceutically acceptable carrier comprising between from about 0.01 mg/kg week to about 1 mg/kg/week of the compound of Formula (I); and, more preferably, from about 0.2 mg/kg/week to about 0.5 mg/kg/week of the conjugate, or from about 0.5 mg/kg/month to about 3 mg/kg/month, and more preferred from about 1 mg/kg/month to about 2 mg/kg/month of the conjugate and may be constituted into any form suitable for the mode of administration selected. The dosages, however, may be varied depending upon the requirement of the subjects, the severity of the condition being treated, and the length of time or titration schedule necessary for a particular dosage regime. For example, for chronic diseases such as an autoimmune disease or for use in immune suppression for transplantation, the dosage regime may be acute or chronic administration, or both, and the pharmaceutical compositions are formulated using doses and pharmaceutical excipients that are tailored for such long-term administration.

Advantageously, the compositions for use in the methods of the invention are in unit dosage forms such as tablets, pills, capsules, dry powders for reconstitution or inhalation, granules, lozenges, prefilled syringes of parenteral solutions or suspensions, metered aerosol or liquid sprays, drops, ampoules, autoinjector devices or suppositories for administration by oral, intranasal, sublingual, intraocular, transdermal, parenteral, rectal, vaginal, dry powder inhaler or other inhalation or insufflation means. Parenteral solutions may also be provided in bulk ampoules or vials wherein the desired amount of drug may be withdrawn for dosing at desired intervals. Alternatively, the composition may be presented in a form suitable for less frequent administration; for example at intervals of every two weeks (bi-weekly), or once per month or at six-week intervals, formulations may be adapted to provide a subcutaneous injection or depot preparation for intramuscular injection.

For preparing solid pharmaceutical compositions for use in the methods of the invention such as tablets, the principal active ingredient is mixed with a pharmaceutical carrier, e.g., conventional tableting ingredients such as diluents, binders, adhesives, disintegrants, lubricants, antiadherents and glidants. Suitable diluents include, but are not limited to, starch (i.e. corn, wheat, or potato starch, which may be hydrolized), lactose (granulated, spray dried or anhydrous), sucrose, sucrose-based diluents, dextrose, inositol, mannitol, sorbitol, microcrystalline cellulose (i.e. AVICEL™ microcrystalline cellulose, FMC Corp. (Philadelphia, Pa.), dicalcium phosphate, calcium sulfate dihydrate, calcium lactate trihydrate and the like. Suitable binders and adhesives include, but are not limited to acacia gum, guar gum, tragacanth gum, sucrose, gelatin, glucose, starch, and cellulosics (i.e. methylcellulose, sodium carboxymethylcellulose, ethylcellulose, hydroxypropylmethylcellulose, hydroxypropylcellulose, and the like), water soluble or dispersible binders (e.g., alginic acid and salts thereof, magnesium aluminum silicate, hydroxyethylcellulose (such as TYLOSE™, Hoechst Celanese, Dallas, Tex.), polyethylene glycol, polysaccharide acids, bentonites, polyvinylpyrrolidone, polymethacrylates and pregelatinized starch) and the like. Suitable disintegrants include, but are not limited to, starches (corn, potato, etc.), sodium starch glycolates, pregelatinized starches, clays (magnesium aluminum silicate), celluloses (such as crosslinked sodium carboxymethylcellulose and microcrystalline cellulose), alginates, pregelatinized starches (i.e. corn starch, etc.), gums (i.e. agar, guar, locust bean, karaya, pectin, and tragacanth gum), cross-linked polyvinylpyrrolidone and the like.

Suitable lubricants and antiadherents include, but are not limited to, stearates (magnesium, calcium and sodium), stearic acid, talc waxes, stearowet, boric acid, sodium chloride, DL-leucine, carbowax 4000, carbowax 6000, sodium oleate, sodium benzoate, sodium acetate, sodium lauryl sulfate, magnesium lauryl sulfate and the like.

Suitable glidants include, but are not limited to, talc, cornstarch, silica (i.e. CABO-SIL™ silica (Cabot, Boston, Mass.) SYLOID™ silica (W. R. Grace/Davison), and AEROSIL™ Degussa) and the like. Sweeteners and flavorants may be added to chewable solid dosage forms to improve the palatability of the oral dosage form. Additionally, colorants and coatings may be added or applied to the solid dosage form for ease of identification of the drug or for aesthetic purposes. These carriers are formulated with the pharmaceutical active to provide an accurate, appropriate dose of the pharmaceutical active with a therapeutic release profile.

Generally these carriers are mixed with conjugate to form a solid preformulation composition containing a homogeneous mixture of the conjugates for use in the present invention. Generally the preformulation will be formed by one of three common methods: (a) wet granulation, (b) dry granulation and (c) dry blending. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from about 0.1 mg to about 500 mg of the active ingredient of the present invention. The tablets or pills containing the novel compositions may also be formulated in multilayer tablets or pills to provide a sustained or provide dual-release products. For example, a dual release tablet or pill can comprise an inner dosage and an outer dosage component, the latter being in the form of an envelope over the former. The two components can be separated by an enteric layer, which serves to resist disintegration in the stomach and permits the inner component to pass intact into the duodenum or to be delayed in release. A variety of materials can be used for such enteric layers or coatings, such materials including a number of polymeric materials such as shellac, cellulose acetate (i.e. cellulose acetate phthalate, cellulose acetate trimetllitate), polyvinyl acetate phthalate, hydroxypropyl methylcellulose phthalate, hydroxypropyl methylcellulose acetate succinate, methacrylate and ethylacrylate copolymers, methacrylate and methyl methacrylate copolymers and the like. Sustained release tablets may also be made by film coating or wet granulation using slightly soluble or insoluble substances in solution (which for a wet granulation acts as the binding agents) or low melting solids a molten form (which in a wet granulation may incorporate the active ingredient). These materials include natural and synthetic polymers waxes, hydrogenated oils, fatty acids and alcohols (i.e. beeswax, carnauba wax, cetyl alcohol, cetylstearyl alcohol, and the like), esters of fatty acids metallic soaps, and other acceptable materials that can be used to granulate, coat, entrap or otherwise limit the solubility of an active ingredient to achieve a prolonged or sustained release product.

The liquid forms comprising the α4β1-selective conjugates for use in the present invention may be incorporated for administration orally or by injection. These forms include, but are not limited to aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable suspending agents for aqueous suspensions, include synthetic and natural gums such as, acacia, agar, alginate (i.e. propylene alginate, sodium alginate and the like), guar, karaya, locust bean, pectin, tragacanth, and xanthan gum, cellulosics such as sodium carboxymethylcellulose, methylcellulose, hydroxymethylcellulose, hydroxyethylcellulose, hydroxypropyl cellulose and hydroxypropyl methylcellulose, and combinations thereof, synthetic polymers such as polyvinyl pyrrolidone, carbomer (i.e. carboxypolymethylene), and polyethylene glycol; clays such as bentonite, hectorite, attapulgite or sepiolite; and other pharmaceutically acceptable suspending agents such as lecithin, gelatin or the like. Suitable surfactants include but are not limited to sodium docusate, sodium lauryl sulfate, polysorbate, octoxynol-9, nonoxynol-10, polysorbate 20, polysorbate 40, polysorbate 60, polysorbate 80, polyoxamer 188, polyoxamer 235 and combinations thereof. Suitable deflocculating or dispersing agent include pharmaceutical grade lecithins. Suitable flocculating agent include but are not limited to simple neutral electrolytes (i.e. sodium chloride, potassium, chloride, and the like), highly charged insoluble polymers and polyelectrolyte species, water soluble divalent or trivalent ions (i.e. calcium salts, alums or sulfates, citrates and phosphates (which can be used jointly in formulations as pH buffers and flocculating agents). Suitable preservatives include but are not limited to parabens (i.e. methyl, ethyl, n-propyl and n-butyl), sorbic acid, thimerosal, quaternary ammonium salts, benzyl alcohol, benzoic acid, chlorhexidine gluconate, phenylethanol and the like. There are many liquid vehicles that may be used in liquid pharmaceutical dosage forms, however, the liquid vehicle that is used in a particular dosage form must be compatible with the suspending agent(s). For example, nonpolar liquid vehicles such as fatty esters and oils liquid vehicles are best used with suspending agents such as low HLB (Hydrophile-Lipophile Balance) surfactants, stearalkonium hectorite, water insoluble resins, water insoluble film forming polymers and the like. Conversely, polar liquids such as water, alcohols, polyols and glycols are best used with suspending agents such as higher HLB surfactants, clays silicates, gums, water soluble cellulosics, water soluble polymers and the like. For parenteral administration, sterile suspensions and solutions are desired. Liquid forms useful for parenteral administration include sterile solutions, emulsions and suspensions. Isotonic preparations which generally contain suitable preservatives are employed when intravenous administration is desired. Suitable parenteral preservatives include phenol, benzyl alcohol, phenoxyethanol, methylparaben or propylparaben and combinations thereof.

In general the compound of Formula (I) may be dissolved in sterile saline or buffered sterile saline at a physiologically acceptable pH that is compatible with the tissue or fluid into which the drug solution is injected or infused, optionally including a suitable preservative.

The α4β1-selective conjugates can also be administered in the form of liposome delivery systems, such as small unilamellar vesicles, large unilamellar vesicles, multilamellar vesicles and the like. Liposomes can be formed from a variety of phospholipids, such as cholesterol, stearylamine, phosphatidylcholines and the like.

A therapeutically effective amount of the conjugate drug is ordinarily supplied at a dosage level of from about 0.1 mg/kg to about 300 mg/kg of body weight per day. Preferably, the range is from about 0.2 mg/kg to about 10 mg/kg of body weight per day; and, most preferably, from about 0.5 mg/kg to about 1.0 mg/kg of body weight per day. Advantageously, a compound of the present invention may be administered in a single weekly, bi-weekly or monthly dose or the total daily dosage may be administered in divided doses of two, three or four times daily.

Optimal dosages to be administered may be readily determined by those skilled in the art, and will vary with the particular compound used, the mode of administration, the strength of the preparation, and the advancement of the disease condition. In addition, factors associated with the particular subject being treated, including subject age, weight, diet and time of administration, will result in the need to adjust the dose to an appropriate therapeutic level.

### General Synthetic Methods

The α4β1-selective conjugates for use in the methods of the present invention can be synthesized in accordance with the general synthetic methods, and as illustrated more particularly in Example 1. Such example is merely an illustration, and methods for production of the α4β1-conjugates for use in the present invention are not to be limited by the chemical reactions and conditions described.

The components of the α4β1-selective conjugates can be provided in the form of pharmaceutically acceptable salts. For use in medicine, the salts of the compounds are “pharmaceutically acceptable salts” such as those disclosed in Ref. International J. Pharm., 1986, 33, 201-217 and J. Pharm. Sci., 1997 (January), 66, 1,1). Other salts may, however, be employed in the preparation of compounds used in the conjugates of the invention.

### Conditions Amenable to Treatment Using the Compounds of the Invention

The methods of the present invention are useful in the treatment of various diseases, disorders and physiological effects that are mediated at least in part through α4β1 integrin signaling.

In certain aspects, the methods of the invention are used to treat autoimmune disorders. A substantial minority of the population suffers from autoimmune diseases, which are often chronic, debilitating, and life-threatening. It has been estimated that autoimmune diseases are among the ten leading causes of death among women in all age groups up to 65 year. There are more than eighty illnesses caused by autoimmunity, as described in Noel R. Rose and Ian R. MacKay, “The Autoimmune Diseases” fourth edition, which is incorporated herein by reference. Such disease conditions include, by way of example, asthma, Alzheimer's disease, atherosclerosis, AIDS dementia, diabetes (including acute juvenile onset diabetes), inflammatory bowel disease (including ulcerative colitis and Crohn's disease), multiple sclerosis, rheumatoid arthritis, tissue transplantation, tumor metastasis, meningitis, encephalitis, stroke, and other cerebral traumas, nephritis, retinitis, Sjogren's disease, atopic dermatitis, psoriasis, myocardial ischemia and acute leukocyte-mediated lung injury such as that which occurs in adult respiratory distress syndrome.

In a specific aspect, the methods of the invention are used to treat multiple sclerosis. The term “multiple sclerosis” as used herein includes all aspects of the disease, including but not limited to primary progressive multiple sclerosis (PPMS), secondary progressive multiple sclerosis (SPMS) and relapsing forms of multiple sclerosis (RFMS), which encompasses relapsing remitting multiple sclerosis and all other relapsing forms of the disease.

In other aspects, the methods of the invention are used to treat inflammatory disease. Inflammatory diseases such as atherosclerosis and restenosis, which both affect arterial blood vessels, result from a chronic inflammatory response in the walls of arteries. Atherosclerosis is the leading cause of cardiovascular disease, and the accumulation and continued recruitment of leukocytes are associated with the development of ‘vulnerable’ atherosclerotic plaques, which are prone to rupture, leading to thrombosis, myocardial infarction or stroke. Plaque macrophages account for the majority of leukocytes in plaques, and are believed to differentiate from monocytes recruited from circulating blood via α4β1-mediated interactions. Woollard K J and Geissmann F, Nat Rev Cardiol. 2010 February; 7 (2):77-86. Epub 2010 Jan. 12. Restenosis, an inflammatory response following intervention such as angioplasty, results in the re-narrowing of a coronary artery. This typically occurs within 3-6 months in 40-50% of patients who have angioplasty, leading to significant pathologies, including myocardial infarction and ischemias.

Both autoimmune and inflammatory diseases arise through aberrant reactions of the human adaptive or innate immune systems. In autoimmunity, the patient's immune system is activated against the body's own cells and/or proteins. In inflammatory diseases, the pathology is due to the overreaction of the immune system, and its subsequent downstream signaling. These processes are mediated at least in part through α4β1 signaling, and thus selective inhibition of α4β1 has a tremendous array of applications in therapeutic intervention.

Other exemplary autoimmune and/or inflammatory disease conditions which may be treated using conjugates and compositions of the present invention include, but are not limited to, inflammatory conditions such as erythema nodosum, allergic conjunctivitis, optic neuritis, uveitis, allergic rhinitis, ankylosing spondylitis, psoriatic arthritis, vasculitis, Reiter's syndrome, systemic lupus erythematosus, progressive systemic sclerosis, polymyositis, dermatomyositis, Wegner's granulomatosis, aortitis, sarcoidosis, lymphocytopenia, temporal arteritis, pericarditis, myocarditis, congestive heart failure, polyarteritis nodosa, hypersensitivity syndromes, allergy, hypereosinophilic syndromes, Churg-Strauss syndrome, chronic obstructive pulmonary disease, hypersensitivity pneumonitis, chronic active hepatitis, interstitial cystitis, autoimmune endocrine failure, primary biliary cirrhosis, autoimmune aplastic anemia, chronic persistent hepatitis and thyroiditis.

In other aspects, the methods of the invention are used to treat cell proliferative disorders. α4β1 integrins have been shown to promote the homing of hematopoietic progenitor cells to A4B1 and fibronectin found on both actively healing and tumor-associated blood vessels. These progenitor cells are drawn to sites of active blood vessel formation in tumors, but not to normal tissues, due to the presence of α4β1 in the tumor vessels. Blocking α4β1 with a composition according to the methods of the invention can prevent the progenitor cells from sticking to blood vessels, from migrating to newly formed vessels, and from changing into different cell types. Disrupting the α4β1-mediated homing process is thus useful in suppressing new blood vessel formation in tumors.

Hematological disorders, and in particular leukemias and multiple myeloma, are also amendable to treatment using the method of the present invention. More specifically, cell proliferative disorders such as acute lymphoblastic leukemia (ALL), acute myelogenous leukemia (AML), chronic myelogenous leukemia (CML), and chronic lymphocytic leukemia (CLL).

In other aspects of the invention, methods are provided for reducing the level of damage caused by injuries that involve invasive leukocyte activity. For example, the extent of disability caused by spinal cord injury (SCI) relates to secondary tissue destruction arising partly from an α4β1-mediated intraspinal influx of neutrophils and monocyte/macrophages after the initial injury. Methods to prevent such neutrophil and monocyte/macrophage influx can significantly reduce oxidative activity in injured cord and improve functional recovery correlated with spared myelin-containing white matter. Fleming J C et al, Exp Neurol. 2008 December; 214 (2):147-59. Epub 2008 May 1. Methods of inhibiting α4β1 and in particular early intervention with the compositions of the invention following injury can significant neurological as a neuroprotective strategy.

### EXAMPLES

The following examples are put forth so as to provide those of ordinary skill in the art with a complete disclosure and description of how to make and use the present invention, and are not intended to limit the scope of what the inventors regard as their invention, nor are they intended to represent or imply that the experiments below are all of or the only experiments performed. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees centigrade, and pressure is at or near atmospheric.

### Example 1

### Production of the Formula (I) Conjugate

The compound of Formula (I) that is used in the preferred methods of the invention was synthesized using the following components:

The 40 kDa 3-arm PEG alcohol (0.25 g, 0.00625 mmol, NOF Corp. Japan) and the small molecule inhibitor 2-(3,3-dimethyl-1-(pyridin-3-ylsulfonyl)pyrrolidine-2-carboxamido)-3-(4-(2-oxo-1H-imidazo[4,5-b]pyridin-3(2H)-yl)phenyl)propanoic acid (0.04 g, 0.056 mmol): along with triphenylphosphine (0.025 g, 0.094 mmol). These components were dried by azeotropic distillation from toluene (5 mL). Half of the volume was distilled over (2.5 mL), and the mixture was cooled to room temperature. CH₂Cl₂ (0.5 mL) was added to make the reaction homogeneous. Diethylazodicarboxylate (0.015 mL, 0.094 mmol) was added drop-wise and the reaction stirred for 48 hours. HPLC Method C showed the complete disappearance of the starting PEG alcohol. The reaction was concentrated in vacuum to yield a t-butyl ester as a white solid. This resulting t-butyl ester is Formula (I).

Likewise ELN 476063, a conjugate of a pan alpha4 beta1 and alpha beta7 inhibitor was made by conjugating the small molecule inhibitor, 2-(2-(diethylamino)-5-(1-oxo-1-(pyrrolidin-1-yl)butan-2-yl)pyrimidin-4-ylamino)-3-(4-(2-oxo-1H-imidazo[4,5-b]pyridin-3(2H)-yl)phenyl)propanoic acid was coupled under Mitsonobu conditions as described for the compound of Formula (I) above.

Detailed methods of making the small molecule inhibitors are provided in published United States application no. US20070037804. Detailed methods of making conjugates with drug molecules such as and including Formula (I) and ELN 476063 are disclosed in published United States application nos. US20070021555 and US20080031848, all of which are incorporated herein in by reference in their entireties.

### Example 2

### In Vitro Potency Studies

The potency of the compound of Formula (I) was determined using several assays that independently measure α4-dependent ligand interaction, specifically: 1) the ability of the compound of Formula (I) to compete with the binding of an α4β1-specific multivalent ligand, 2) inhibition of the α4β1-dependent adhesion of human lymphocytic cells (Jurkat™ cell line) to human VCAM-1; 3) inhibition of the α4β1-dependent adhesion of human lymphocytic cells (Jurkat™ TM cell line) to fibronectin; and 4) inhibition of the binding of human serum FN to activated α4β1 on Jurkat™ TM cells. Each of these assays confirmed the potency of Formula (I) based on α4β1 binding.

### Multivalent Competition Assay

Potency of Formula (I) was evaluated in a lymphocyte binding competition assay using an α4β1-specific multivalent ligand. This ligand is composed of a nonspecific antibody serving as a carrier for several small molecules with high affinity for α4β1. This multivalent ligand was prepared as follows.

A non-specific antibody was dissolved in a non-nucleophilic buffer, and treated with EDC and sulfo-NHS, thereby activating aspartate and glutamate side chains on the surface of the antibody. Thereafter, a small molecule inhibitor of α4β1 integrin, bearing a primary amino group in an area of the molecule not important for binding to α4β1 integrin, was added to the mixture. After allowing the conjugation reaction to proceed for an appropriate period, the reaction mixture was quenched by addition of 2-hydroxyethylamine. Then, the non-specific antibody, now displaying multiple copies of the small molecule α4β1 inhibitor, was separated from low molecular weight contaminants by dialysis. The multivalent ligand showed high avidity to Jurkat™ cells, which display α4β1 integrin, and competition of this interaction is a means for discriminating the potencies of very potent α4β1-inhibiting compounds. Thus, this assay is a very stringent assay used to characterize the potency of the Formula (I) molecule. Binding of Formula (I) to α4β1 was demonstrated by a competition binding assay demonstrating the ability of Formula (I) to displace the multivalent ligand on human lymphocytes.

Jurkat™ cells were incubated in assay buffer with a titration of Formula (I) in the presence of the multivalent ligand diluted at 1:400 for 30 minutes at room temperature. Unbound reagent was then removed by several wash steps in which the cells were pelleted in a Beckman tabletop centrifuge at 300× gravity for 5 minutes and then resuspended in fresh buffer. Remaining bound antibody multivalent ligand was detected by incubating the cells with Goat F(ab)′₂ anti-mouse IgG(Fc)-Phycoerythrin (Beckman-Coulter Inc., Brea Calif.) for 30 minutes at 4° C., followed by washing and FACS analysis.

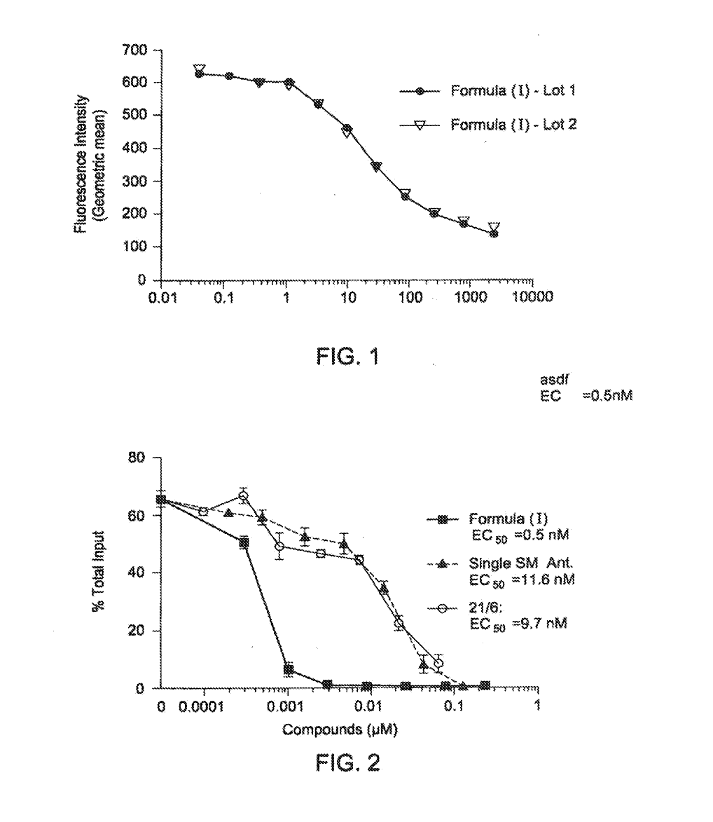

The compound of Formula (I) displayed significant displacement of the binding of the multivalent ligand. The inhibition was reproducible and consistent between lots tested (See FIG. 1), with the molecule, s displaying an IC50 of 18.8 nM and 17.7 nM. The potency of Formula (I) was not altered in the presence of 100% serum from different species, demonstrating that Formula (I) did not bind to serum proteins (data not shown).

α4β1-Dependent Adhesion: Jurkat™ Adhesion to rs-hu-VCAM-1

Jurkat™ lymphoma T cells express high levels of α4β1 integrin and strongly adhere to VCAM-1 in an α4 integrin-dependent fashion. An assay to assess the inhibition of this adhesion was also used to determine potency of the Formula (I) molecule. The ability of Formula (I) to prevent adhesion was compared to the inhibition using Formula (III) (a single small molecule component of Formula (I)), and an anti-α4 antibody, 21/6.

A 96-well plate (Costar 3590) was coated with rs-hu-VCAM-1 (R&D Systems, Santa Cruz, Calif.) using 0.25 μl/well in PBS++ (PBS+1 mM CaCl₂+1 mM MgCl₂) and incubated overnight at 4° C. Following incubation, the plate was blocked with assay buffer (20 mM Hepes, 140 mM NaCl, 1 mM CaCl₂, 1 mM MgCl₂ and 0.3% BSA) for 30 minutes at RT and washed 3 times with assay buffer. Fluorescently labeled Jurkat™ cells (Calcein fluorescent dye, Molecular Probes, Carlsbad, Calif.) were added to wells of a 96-well FlexiPlate (Falcon 353911) at 10⁵ cells/well (final volume: 100 μl/well) in assay buffer. Following addition of the conjugate, the compound or the antibody at various concentrations, the cells were incubated for 30 minutes at RT. Cells were then transferred to the rs-hu-VCAM-1-coated plate and binding was allowed for 30 minutes at RT. The plate was washed 3 times with assay buffer and read for fluorescence (Applied Biosystems, CytoFluor series 4000, Foster City, Calif.).

Formula (I) strongly inhibited the α4β1-dependent adhesion of Jurkat™ cells to human VCAM-1 with an EC50 value of 0.5 nM (FIG. 2). Interestingly, although there are three α4β1 antagonist small molecules per compound of Formula (I), Formula (I) appeared 23-fold more potent than Formula (III) (EC50 of 0.5 nM vs 11.6 nM, respectively).

### α4β1-Dependent Adhesion: Jurkat™ Cell Adhesion to Human Fibronectin

Jurkat™ cells adhere to fibronectin (FN) in an μ4β1-dependent fashion. Inhibition of this adhesion using Formula (I), Formula (III) and an anti-α4 antibody, 21/6, was also used to determine potency of the Formula (I) molecule.

A 96-well plate (Costar 3590) was coated with fibronectin (FN) (Calbiochem) at 0.6 mg/well in PBS++ (PBS+1 mM CaCl₂+1 mM MgCl₂) overnight at 4° C. Then the plate was blocked with assay buffer (20 mM Hepes, 140 mM NaCl, 1 mM CaCl₂, 1 mM MgCl₂, and 0.3% BSA) for 30 minutes at RT and washed 3 times with assay buffer. Fluorescently-labeled Jurkat™ cells (calcein fluorescent dye, Molecular Probes, C-1430, used according to manufacturer procedure) were added to wells of a 96-well FlexiPlate (Falcon 353911) at 10⁵ cells/well (final volume: 100 μL/well) in assay buffer+10 μg/mL TS2/16 antibody against β1 integrin that activates α4β1. 5 μg/mL of an inhibitory antibody against α5 integrin (BD Pharmingen, Franklin Lakes, N.J.) is added to the buffer to prevent α5-dependent adhesion to FN. Following addition of compounds or anti-α4 antibodies (21/6, GG5/3, or HP2/1) at various concentrations, the cells were incubated for 30 minutes on wet ice. Cells were then transferred to the FN coated plate and binding was allowed for 35 minutes at RT. The plate was washed 3 times with assay buffer and read for fluorescence (Applied Biosystems, CytoFluor Series 4000).

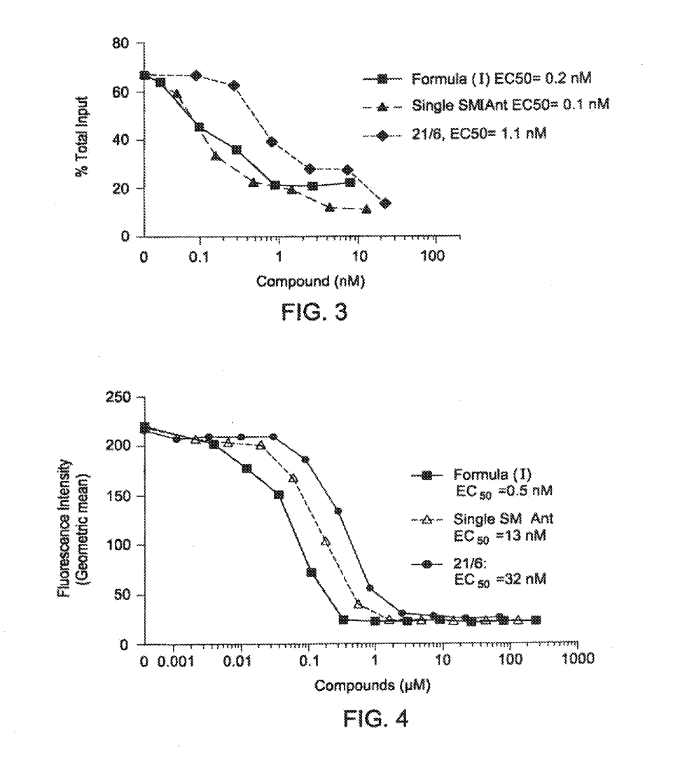

The interaction of FN and α4β1 is less stringent than the VCAM-1 interaction as illustrated by the shift of 21/6 EC50 from 9.7 nM in the VCAM-1 assay to 1.1 nM in the FN assay (see FIG. 3). Formula (I) inhibited FN adhesion with an EC50 of 0.2 nM, similar to its small molecule component (0.1 nM). The two compounds Formula (I) and Formula (III) are very potent compounds and therefore have reached the sensitivity of the assay and potency difference cannot be measured anymore.

### α4β1-FN Capture Assay in Human Serum

The α4β1 receptors on Jurkat™ cell surfaces can capture, in solution, the endogenous FN present in serum. Inhibition of this monovalent interaction was also used as potency evaluation of Formula (I).

Log-phase Jurkat™ cells were incubated in a 96-well FlexiPlate under the following conditions: 10⁵ cells/50 μl/well in assay buffer (20 mM Hepes, 140 mM NaCl, 1 mM CaCl₂, 1 mM MgCl₂, and 0.3% BSA) containing 50% human serum, 2× (20 μg/ml) of an inhibitory antibody against α5 integrin (BD Pharmingen, Franklin Lakes, N.J.) and 2× compounds or antibodies at a range of concentrations for 30 minutes at RT. An equal volume of assay buffer containing 50% human serum, the antibody 15/7 (2×, 20 μg/ml) and MnCl₂ (2×, 3 mM) was then added to each well. These reagents served as α4 integrin activating agents, allowing the receptor to capture FN from the serum. The plate was then incubated for another 30 minutes at RT. Cells were then washed twice with cold assay buffer and incubated with chicken anti-human FN antibody (American Research Products, Palos Verde, Calif.) at 15 μg/mL in cold assay buffer for 30 minutes on ice in the dark. Cells were then washed once and resuspended in 300 μl cold assay buffer for FACS analysis (Becton-Dickinson, Franklin Lakes, N.J.).

Formula (I) inhibited FN capture in 50% human serum at an EC50 of 0.05 nM (FIG. 4). In this assay, Formula (III) was still less potent than Formula (I) but only by 2.6-fold (unlike the 23-fold difference observed in the VCAM-1 adhesion assay). Formula (I) was about 6-fold more potent than the anti-α4 antibody 21/6 (EC50=0.32 nM). This assay, measuring the interaction of the receptor with a monovalent soluble ligand, has a lower stringency than the VCAM-1 adhesion assay, which involves multivalent interactions.

### Example 3

### In Vitro Selectivity Studies

To characterize its specificity, Formula (I) was tested in several cell adhesion assays that are dependent on integrins other than α4β1 and in the multivalent ligand assay with α9β1, the integrin that is most closely related to α4β1. The specificity of Formula (I) was assessed by examining its activity in four non-α4β1 integrin-dependent assays: 1) an α4β7-dependent adhesion to A4β7; 2) an aLβ2 (LFA-1)-dependent adhesion to ICAM-1; 3) an α5β1-dependent adhesion to human fibronectin (FN); and 4) a multivalent competition on α9β1 cells. These are each described briefly as follows.

### α4β7-Dependent Adhesion: 8866 Cell Adhesion to A4β7-Fc

Wells of a 96-well plate (Costar 3590) were coated with a mouse antibody against Human Fc (Sigma. I-6260) by incubating them with a 1:300 dilution of the antibody in H/S++ (20 mM Hepes, 140 mM NaCl, 1 mM CaCl₂, 1 mM MgCl₂) for 1 hr at RT. The plate was blocked with assay buffer (20 mM Hepes, 140 mM NaCl, 1 mM CaCl₂, 1 mM MgCl₂ and 0.3% BSA) for 1 hr at RT and then washed 3 times with the same buffer. Recombinant human A4β7-Fc in assay buffer (20 mM Hepes, 140 mM NaCl, 1 mM CaCl₂, 1 mM MgCl₂ and 0.3% BSA) was then added to the wells at 30 ng/well and incubated overnight at 4° C. The plate was then washed 3 times in assay buffer. Fluorescently-labeled 8866 cells (calcein fluorescent dye, Molecular probes. C-1430, used according to manufacturer procedure) were added to wells of a 96-well FlexiPlate (Falcon 353911) at 10⁵ cells/well (final volume: 100 μl/well) in assay buffer. Following addition of compounds or anti-α4 antibodies (21/6, GG5/3 or HP2/1) at various concentrations, the cells were incubated for 30 minutes at RT. Cells were then transferred to the A4β7-Fc coated plate and binding was allowed for 30 minutes at RT. The plate was washed 3 times with assay buffer and read for fluorescence (Applied Biosystems, CytoFluor series 4000, Foster City, Calif.).

### αLβ2-Dependent Adhesion: 8866 Cell Adhesion to ICAM-1-Fc

A 96-well plate (Costar 3590) was coated with mouse ascites anti-Hu Fc (Sigma-Aldrich, St. Louis, Mo.) at 1:300 in H/S++, 1 hr at RT. The plate was blocked with assay buffer (20 mM Hepes, 140 mM NaCl, 1 mM CaCl₂, 1 mM MgCl₂ and 0.3% BSA) for 1 hr at RT and washed 3 times with the same buffer. (rhuman-ICAM-1-Fc (R&D Systems, Santa Cruz, Calif., 720-IC) at 40 ng/well in assay buffer was then captured overnight at 4° C. and the plate was washed again 3 times in assay buffer. Calcein labeled 8866 cells, at 10⁵ cells/well in assay buffer (final volume: 100 μl/well), were pre-incubated in a 96-well FlexiPlate with 50 ng/mL PMA and with a concentration range of compound, for 30 minutes at RT. An anti-αLβ2 (Pharmingen, 555381) at 10 μg/mL was also used as a positive control to inhibit the αLβ2-dependent binding. Cells were then transferred to the ICAM-1-Fc coated plate and binding was allowed for 35 minutes at 37° C., 5% CO₂ (incubator). The plate was washed 3 times with assay buffer and read for fluorescence (Applied Biosystems, CytoFluor series 4000).

### α5β1-Dependent Adhesion: THP-1 Cell Adhesion to Human FN

A 96-well plate (Costar 3590) was coated with FN (Calbiochem, Gibbstown, N.J.) at 0.6 μl/well in PBS++ (PBS+1 mM CaCl₂+1 mM MgCl₂) overnight at 4° C. Then the plate was blocked with assay buffer (20 mM Hepes, 140 mM NaCl, 1 mM CaCl₂, 1 mM MgCl₂, and 0.3% BSA) for 30 minutes at RT and washed 3 times with assay buffer. Calcein labeled THP-1 cells, at 10⁵ cells/well in assay buffer (final volume: 100 μL/well), were pre-incubated in a 96-well FlexiPlate with 10 μg/mL 21/6, 10 μg/ml TS2/16, and with a concentration range of compound, for 30 minutes at RT. An anti-α5 antibody (Pharmingen Franklin Lakes, N.J.) at 5 μg/mL was also used as a positive control to inhibit the α5β1 dependent binding. Cells were then transferred to the FN-coated plate and binding was allowed for 30 minutes at RT. The plate was washed 3 times with assay buffer and read for fluorescence (Applied Biosystems, CytoFluor series 4000).

### Competition Binding to α9β1

Log-phase SW480-expressing α9β1 cells were incubated in a 96-well FlexiPlate (Falcon 353911) under the following conditions: 10⁵ cells/100 μL/well in assay buffer (20 mM Hepes, 140 mM NaCl, 1 mM CaCl₂, 1 mM MgCl₂ and 0.3% BSA), and a small molecule that selectively binds to α9β1 conjugated to an irrelevant mouse antibody. The α9β1 binder were provided at its EC90 concentration (1:400 dilution), and Formula (I) was provided at a range of concentrations. The incubation was performed for 30 minutes at RT. Unbound reagents were then removed by two washes in assay buffer. Remaining bound α9β1 binding molecule were detected by Goat Fab′2 anti Mouse IgG (Fc)-PE (Immunotech, Miami, Fla.) used at a 1:200 dilution in assay buffer for 30 minutes on ice in the dark. Cells were then washed once and resuspended in 300 μl cold assay buffer for FACS analysis (Becton-Dickinson, Franklin Lakes, N.J.).

### Results

Formula (I) had no measurable activity against aLβ2 (LFA-1) and α5β1 integrin, and had measurable activity on α4β7 and α9β1 with an EC50 of 38 nM and 51 nM, respectively. The potency of Formula (I) for α9β1 was within 3-fold of its potency for α4β1 (average MV competition EC50 α4β1=16.5 nM vs EC50 α9β1=51 nM). Results are summarized in Table 1.

Formula (I) demonstrated a >75-fold preference for inhibiting the activity of α4β1 over α4β7; a mild cross-reactivity to α9β1, which is not unexpected based on homology between α4 and α9 and overlapping ligands; and no activity against aLβ2 (LFA-1) and α5β1 integrins. Table 1 summarizes the specificity data:

TABLE 1
Summary of In Vitro Specificity of Formula (I) as Average
EC₅₀ Values in 4 Non-α4β1 Integrin-Dependent Assays

| | Integrin-Specific Assay - EC₅₀ | | | |
|---|---|---|---|---|
| | α4β7 - A4β7 | αLβ2 - ICAM-1 | α5β1-FN | α9β1 MV |
| Matrix | Adhesion | Adhesion | Adhesion | competition |
| H/S++/ | 38 nM | >10 μM | >1 μM | 51 nM |
| 0.3% BSA | (n = 1) | (n = 3) | (n = 1) | (n = 2) |

H/S++ = Hepes/Saline plus calcium and magnesium, assay buffer
BSA = Bovine serum albumin

### Example 4

### Human Pharmacokinetics Using the α4β1-Selective Conjugate

The plasma pharmacokinetic behavior of the α4β1-selective compound of Formula (I) was evaluated.

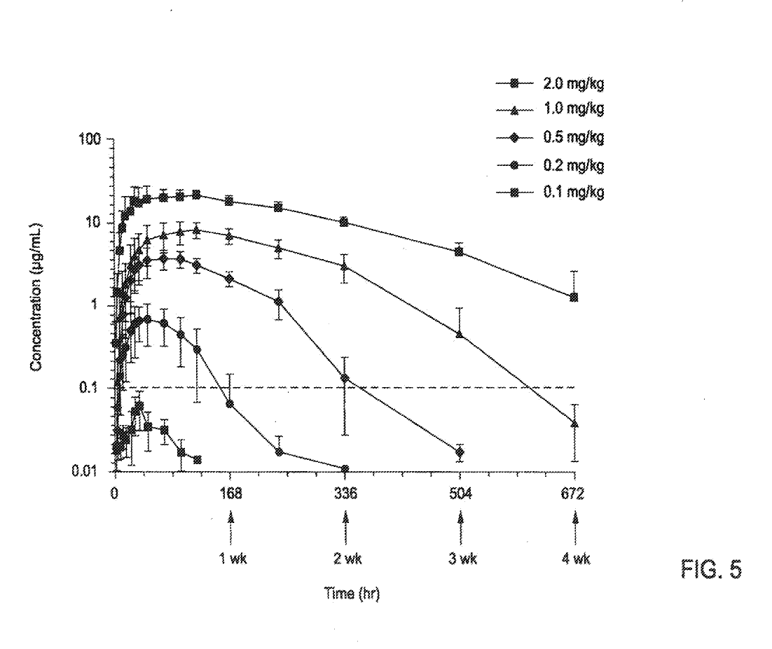

Healthy human subjects between the ages of 18-45 years were enrolled in the study. 39 healthy men and women aged 18-45 years (inclusive) were enrolled in the study. The subjects were divided into six cohorts, and the subjects dosed with 0.05, 0.1, 0.2, 0.5, 1.0 and 2.0 mg/kg of the compound of Formula (I), respectively. Peripheral venous blood samples were drawn from each subject by direct venipuncture into collection tubes. 4.0 ml blood samples were collected from each subject into sodium heparin for measurement of plasma concentrations of the Formula (I) conjugate at the following times: Pre-dose, 2, 4, 8, 12, 16, 24, 30, 36, 48, 72, 96, 120, 168, 240, 336, 504, 672, 1440, and 2160 hours post-dose. Liquid chromatography tandem mass spectrometry (LC/MS/MS) detection, was used to determine Formula (I) plasma concentrations in subjects receiving the conjugate. The summaries of these results are shown in FIG. 5.

The lower limit of quantitation (LLOQ) for Formula (I) was 10.0 ng/mL and the calibration curves were acceptable over a range of 10.0-1000 ng/mL. Noncompartmental pharmacokinetic (PK) parameters were calculated using WinNonlin 5.2 software (Pharsight Corporation, Mountain View, Calif.). Table 2 is a summary of the preliminary PK parameter estimates for the first five cohorts.

TABLE 2
Pharmacokinetic Parameter Estimates

| | | Cohort 2 | Cohort 3 | Cohort 4 | Cohort 5 |
|---|---|---|---|---|---|
| | | 0.1 mg/kg | 0.2 mg/kg | 0.5 mg/kg | 1.0 mg/kg |
| Parameter | Units | n = 7 | n = 8 | n = 8 | n = 8 |
| Cₘₐₓ | μg/mL | 0.062 ± 0.030 | 0.727 ± 0.336 | 3.93 ± 1.03 | 8.48 ± 1.78 |
| Tₘₐₓ | Hr | 34.5 ± 3.0 | 46.8 ± 16.1 | 68.0 ± 23.6 | 132.0 ± 65.7 |
| AUCₗₐₛₜ | μg · hr/mL | 2.8 ± 1.6 | 69.7 ± 41.6 | 666 ± 155 | 2168 ± 317 |
| AUC_{0-inf} | μg · hr/mL | ND | 70.3 ± 41.6 | 667 ± 155 | 2219 ± 295 |
| CL/F | mL/hr/kg | ND | 3.4 ± 1.3 | 0.7 ± 0.2 | 0.4 ± 0.1 |
| Vz/F | mL/kg | ND | 109 ± 42.6 | 44.7 ± 10.1 | 45.2 ± 22.3 |

Abbreviations: AUC_{0-inf} = area under the curve zero to infinity; AUC₀₋ₗₐₛₜ = area under the curve zero to the last measurable concentration; CL/F = clearance uncorrected for bioavailability; Cₘₐₓ = maximal concentration; hr = hour; kg = kilogram; mL = milliliters; μg = microgram; ND = not determined; Tₘₐₓ = time to maximum observed concentration; Vz/F = volume of distribution uncorrected for bioavailability.

At all time points for Cohort 1 (0.05 mg/kg), the concentrations of Formula (I) remained below the LLOQ of 10 ng/mL. No PK parameter estimation was performed for this group. Formula (I) exposure increased in a greater than expected manner for the 0.1, 0.2, 0.5, 1.0 and 2.0 mg/kg dose groups. Elimination half-life is difficult to assess due to the non-linear nature of the concentration versus time curves.

In summary, following Formula (I) subcutaneous (SC) administration, AUC increased in a greater than dose proportional manner, resulting from a dose related prolongation in the elimination half-life of Formula (I). The Cmax also increased in a greater than expected manner. The PK behavior of Formula (I) was characterized by a decrease in systemic clearance and apparent volume of distribution with increasing dose. The initial studies support preferential dosage ranges for Formula (I) of 0.2-0.5 mg/kg per week and 1.0-2.0 mg/kg per month.

### Example 5

### Integrin Receptor Saturation Assays

Peripheral blood lymphocytes were collected from blood samples of the six cohorts of the human trials described in Example 4, and analyzed for percentage saturation of the α4β1 and α4β7 receptor and the total α4 receptor number.

α4β1 integrin saturation, α4β7 integrin saturation and total α4 receptor number was determined on the collected circulating lymphocytes. 2 ml lysing buffer was added to each tube, and the tubes vortexed for 2-4 seconds. They were again incubated at 2-8° C. for 13-17 minutes and covered in aluminum foil to reduce light exposure. At the end of such incubation, the cells were vortexed and centrifuged at 2200 rpm for five minutes at 4-15° C. The supernatant is aspirated and discarded, and the pellet resuspended in 0.5 ml of Ortho's fixative solution [1% formaldehyde in PBS (phosphate-buffered saline), pH 7.6] A 13× solution of Formula (I) and a 13× solution of 15/7 or 2G3 was provided in an assay medium containing 5% by volume of FBS in 1×PBS.

The α4β1 integrin saturation assay was performed with a ligand-induced binding site antibody, 15/7, which recognizes a conformational change in the β chain of the integrin when Formula (I) binds to the heterodimer. This conformational change reveals an epitope that is recognized by a mouse-antihuman antibody. Phycoeruthrin-labeled donkey anti-mouse IgG was used as a reporter. Flow cytometry analysis using a FACSCalibur™ flow cytometer (Becton Dickinson, Franklin Lakes, N.J.) was performed to evaluate percent saturation, and excess Formula (I) added to a different aliquot of the blood sample to determine 100% saturation. mAb13, a commercially available rat monoclonal anti-human antibody to the β1 integrin chain, was added to a different aliquot of blood to obtain 0% saturation. mAb13 abolished binding of 15/7, and was used to establish baseline for the α4β1 assay.

The α4β7 integrin saturation assay was performed with an antibody 2G3 which selectively binds to the β7 chain of the integrin. Whole blood was collected in sodium heparin tubes, and Formula (I) was added to an aliquot of blood to a final concentration of 25 μg/ml to prepare the saturated sample. Formula (I) was added to an aliquot of blood to a final concentration of 5 μg/ml to prepare the sample for background. The test aliquots (including without Formula (I) were then treated 2G3 and binding of Formula I to lymphocytes was detected using F(ab′)₂ anti-mouse IgGPE to detect the bound 2G3. The inhibitory monoclonal antibody FIB27, a commercially available monoclonal anti-human antibody to the β7 integrin chain, was added to a different aliquot of blood to obtain 0% saturation. FIB27 abolished binding of 2G3, and was used to establish baseline for the α4β7 assay.

The PE fluorescence of lymphocytes was analyzed using the FACSCalibur™ flow cytometer, and a mean fluorescent intensity (MFI) was determined for the test samples, the background, and the saturated in both of these assays.

The percent saturation of both α4β1 and α4β7 was calculated by subtracting the test sample measurement minus the determined background measurement, dividing this by the saturation level measurement minus background measurement, and multiplying the divided number by 100.

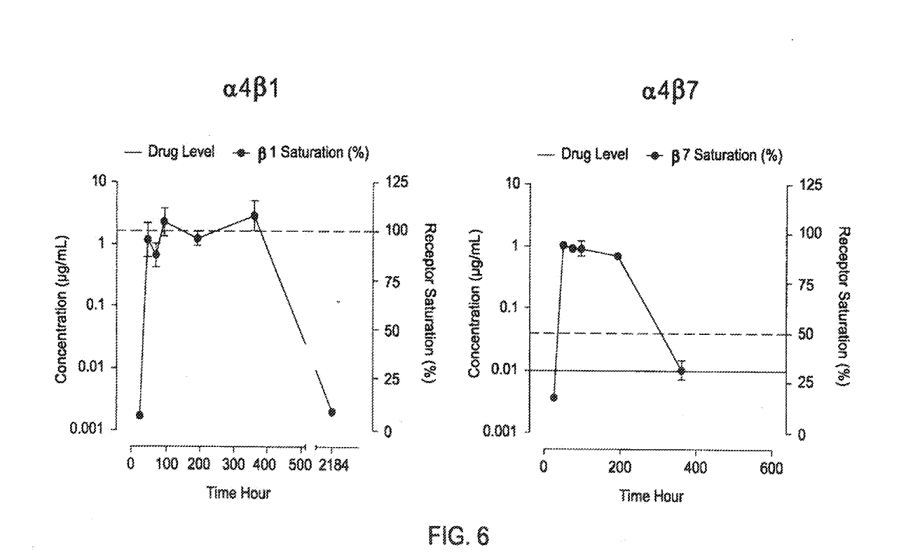

At all dosages, 70-100% saturation of the β1 receptor occurred, however, in Cohorts 4-6, the saturation persisted for up to 14 days (See FIG. 6). Correspondingly, the total number of α4 receptors was decreased with all dosage levels (data not shown). The percentage saturation of α4β7 was investigated beginning with cohort 4 (0.5 mg/kg). At this dosage and higher, saturation of α4β7 was ≧80%. Receptor occupancy of α4β1 and α4β7 appear to be comparable at ≧0.5 mg/kg dosing for more than 1 week. α4β1 saturation persists longer than α4β7 saturation (at 0.5 mg/kg; lower doses not tested for α4β7 saturation).

### Example 6

### Integrin Receptor Down Regulation Following Treatment of Human Subjects with Formula (I)

Peripheral blood lymphocytes were collected from the human cohort receiving 0.5 mg/kg as described in Example 4. 10 μl 9F10-PE (Biolegend, San Diego, Calif.), a mouse anti-α4 integrin antibody that cross reacts with human α4 integrin, was added to the tubes for determination of total anti-α4 down regulation and the tubes vortexed for about two seconds. The tubes were then incubated at 2-8° C. for 30-50 minutes.

2 ml lysing buffer was added to each tube, and the tubes vortexed for 2-4 seconds. They were again incubated at 2-8° C. for 13-17 minutes and covered in aluminum foil to reduce light exposure. At the end of such incubation, the cells were vortexed and centrifuged at 2200 rpm for five minutes at 4-15° C. The supernatant is aspirated and discarded, and the pellet resuspended in 0.5 ml of Ortho's fixative solution [1% formaldehyde in PBS (phosphate-buffered saline), pH 7.6] A mean fluorescent intensity (MFI) was determined for the binding of 9F10-PE, and normalized to the time 0 MFI of the same sample (the baseline) to calculate the % receptor down regulation. The MFI for α4β1 and α4β7 were determined using the normalized MFI from the 15/7 or the 2G3 antibody assays as described in Example 4.

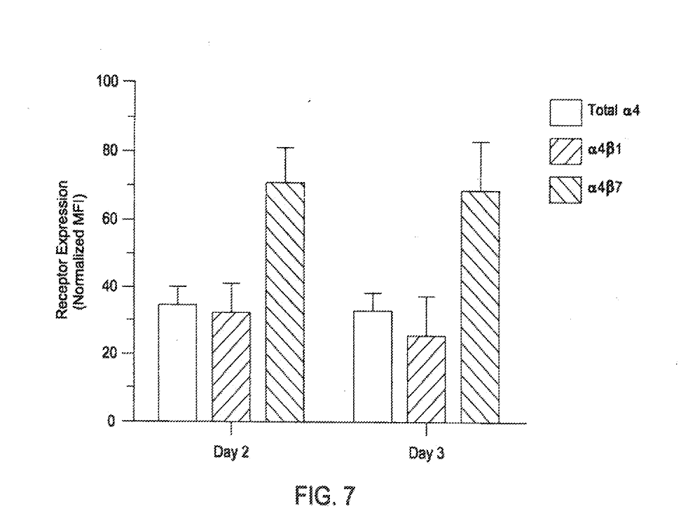

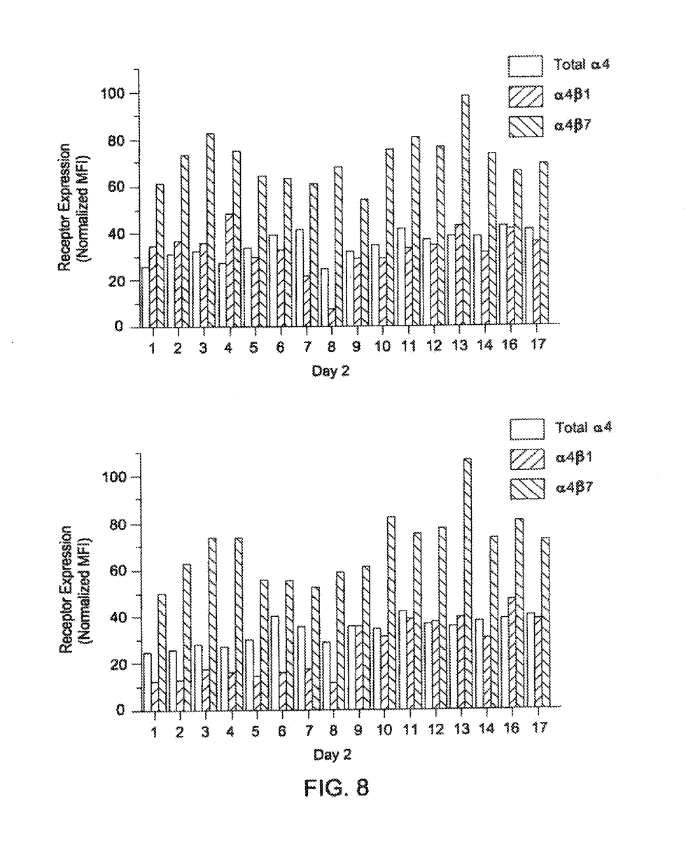

The results of the receptor down regulation in the human trial cohort receiving 0.5 mg/kg dosage of Formula (I) is shown in FIGS. 7 and 8. FIG. 7 shows the averaged down-regulation within the cohort, and FIG. 8 shows the results from individuals receiving this dosage. Total α4 and α4β1 receptor down regulation was correlated. No significant α4β7 receptor down regulation was observed.

### Example 7

### Effect of Formula (I) Dosage on Lymphocyte Count and Receptor Occupancy

The physiological effect of different dosages of Formula (I) in human subjects was compared to that of the conventional α4 targeting therapeutic, Natalizumab (Tysabri™). Natalizumab (Tysabri™) is an anti-α4 antibody that binds to and modulated both α4β1 and α4β7.

Peripheral blood lymphocytes were collected from the various human cohorts receiving single doses of 0.1 mg/kg, 0.2 mg/kg, 0.5 mg/kg, 1.0 mg/kg or 2.0 mg/kg, as described in Example 4, and the lymphocyte counts as a percentage of baseline were measured on Day 6 following the initial dosing. Peripheral venous blood samples were drawn from each subject by direct venipuncture into collection tubes. 4.0 ml blood samples were collected from each subject. The levels of lymphocytes were measured by standard clinical protocols.

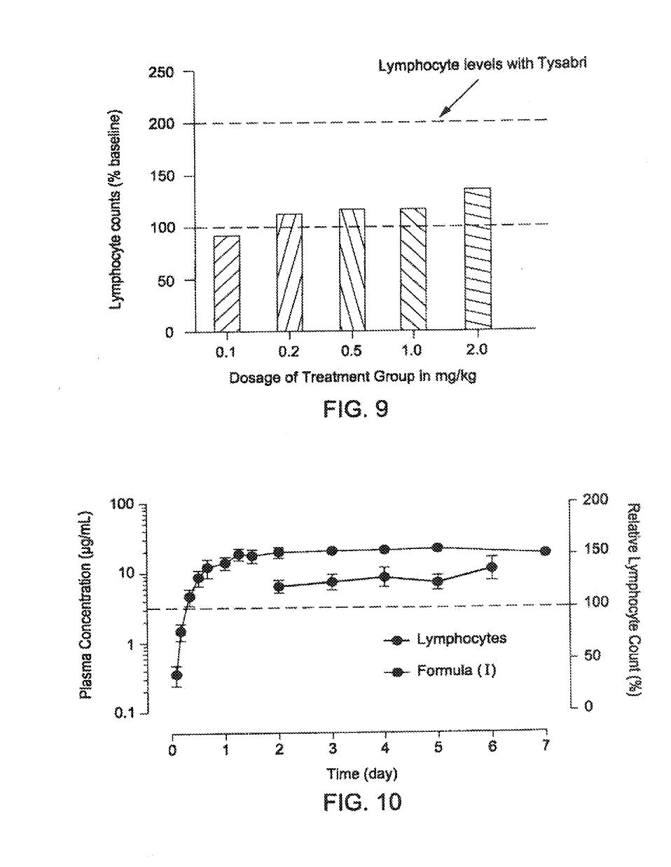

FIG. 9 is a bar graph showing patient lymphocyte counts at different dosages of Formula (I), and FIG. 10 is a line graph illustrating the relationship between plasma concentrations of Formula (I) and relative lymphocyte count in patients. Each of these dosage groups demonstrated a lower level of lymphocyte counts compared to patients receiving comparable levels of Natalizumab (Tysabri™).

### Example 8

### Effect of Formula (I) Dosage on Receptor Saturation

The effect of different doses of Formula (I) on α4β1 and α4β7 receptor saturation, occupancy and expression on lymphocytes was determined in an ascending dose study. The methods for determining receptor saturation were as set forth in Example 5. The levels were followed for 91 days post initial dosing in the different cohorts.

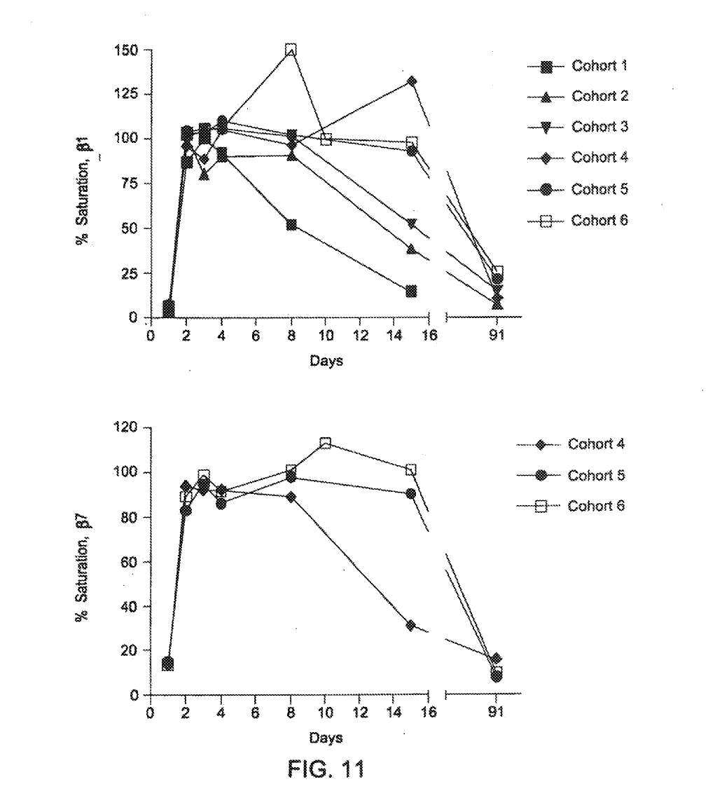

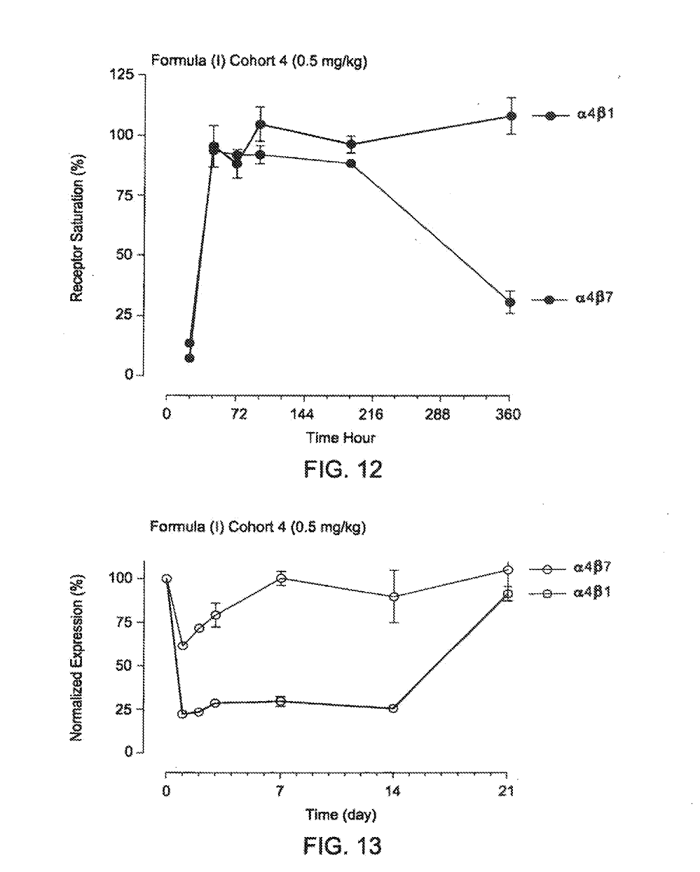

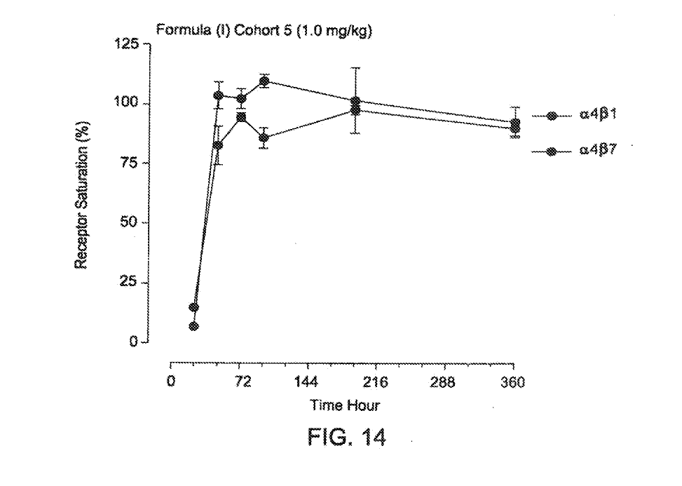

Between 0.1 mg/kg-2 mg/kg, α4β1 receptors on lymphocytes were fully saturated for at least 1 week, as shown in FIG. 11. In contrast, α4β7 receptor saturation returned to baseline by 14 days post-dose. In specific cohorts, this measured level of saturation was shown to be independent of the expression level, indicating that this was a demonstration of selectivity. For example, in Cohort 4, as shown in FIG. 12, the level of α4β1 saturation remained high at 360 post dosing, while the saturation of α4β7 dropped to below 30%. As shown in FIG. 13, the expression levels of α4β7 actually remained higher than that seen for α4β1. The higher dose of Formula (I) given to Cohort 5, 1.0 mg/kg, resulted in a higher saturation level of both α4β1 and α4β7, as shown in FIG. 14. This demonstrates that the level of saturation of the receptors is dose dependent, and can be controlled by appropriate dosage amounts and regimens.

### Example 9

### Dose Selection

The preferred dosage of Formula (I) is that which exhibits efficacy through its modulation of α4β1, but which minimizes any adverse events that may be mediated by α4β7. In order to evaluate the lowest known dose resulting in ˜90% α4β1 receptor saturation, dose ranges and dosing regimens providing discrete levels and durations of α4β1 receptor saturation are determined. This evaluation determines the lowest dose likely to result in acceptable α4β1 receptor saturation while reducing α4β7 saturation. The results from such studies are as summarized in Table 3:

TABLE 3
Pharmacodynamic Outcome at Different Formula (I) Doses

| Dose Level | |
|---|---|
| Regimen | Pharmacodynamic Outcome |
| 0.05 mg/kg | α4β1 Saturation: >90% for ≧3 days; >50% entire study |
| Weekly | α4β7 Saturation: <10% entire study |
| 0.2 mg/kg | α4β1 Saturation: >90% for ≧7 days; >50% for ≧14 days |
| Monthly | α4β7 Saturation: <10% entire study |
| 0.2 mg/kg | α4β1 Saturation: >90% entire study |
| Weekly | α4β7 Saturation: <25% entire study |
| 0.8 mg/kg | α4β1 Saturation: >90% for ≧14 days; >50% entire study |
| Monthly | α4β7 Saturation: >90% for ~7-14 days |
| 1.6 mg/kg | α4β1 Saturation: >90% entire study |
| Monthly | α4β7 Saturation: >90% entire study |

### Example 10

### Selective Inhibition of α4β1-Mediated Cell Adhesion by Formula (I)

The inhibition of α4β1 and α4β7-mediated cell adhesion to α4β1 and α4β7, respectively, by the compound of Formula (I) was examined using a cell-based SRU BIND™ cell adhesion assay (SRU Biosystems, Woburn, Mass.). The compound of Formula (I) was incubated with α4β1+Jurkat cells or α4β7+8866 cells at a range of concentrations. For comparison, ELN476063 was also tested. The compound-cell mixtures were added to the SRU BIND™ assay plates which had been coated with α4β1 or αβ7, and cell adhesion was measured by PVW shift.

Briefly, TiO₂ SRU BIND™ assay plates were coated with anti-human Fc antibody followed by human Fc-α4β1 or Fc-α4β7. Formula (I) and ELN476063, a pegylated small molecule α4-selective integrin inhibitor, were incubated with α4β1+Jurkat cells or α4β7+8866 cells at a range of concentrations. The compound-cell mixtures were then added to the SRU Bind assay plate to initiate cell adhesion which was measured by PVW shift. 100% inhibition of adhesion was defined as the PWV shift observed with cells pre-treated with a saturating concentration of an anti-α4 integrin blocking antibody and 0% inhibition (maximal cell adhesion) was defined as the PWV shift observed with untreated Jurkat or 8866 cells incubated on α4β1 or α4β7 plates, respectively.

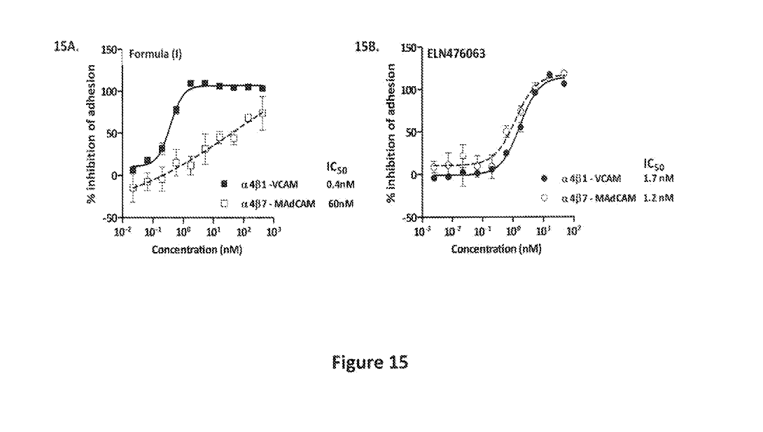

The compound of Formula (I) inhibited the adhesion of α4β1+ cells to VCAM-1 approximately 150-fold more potently than α4β7+ cells to α4β7, with IC50's of 0.4 and 60 nM, respectively (FIG. 15A). In contrast, ELN476063 exhibited equipotent α4β7 of α4β1- and α4β7-mediated cell adhesion to α4β1 and α4β7 with IC50s of 1.7 and 1.2 nM, respectively (FIG. 15B). The compound of Formula (I) inhibited α4β1 adhesion over four-fold more potently than ELN470603.

Additionally, Formula (I) compound inhibited α4β1-mediated adhesion over four-fold more potently than ELN470603, demonstrating Formula (I) compound is a potent and selective inhibitor of α4β1-mediated adhesion, but not α4β7-mediated adhesion.

### Example 11

### Selective Down-Regulation of the Soluble Form of the α4β1 Ligand VCAM-1 Over the α4β7 Ligand MadCAM-1

It has been shown that inhibition of α4 integrins causes the down-regulation of the soluble circulating forms of their ligands, VCAM-1 and MAdCAM-1 (Millonig et al., J. Neuroimmunology October 8; 227 (1-2):190-4. Epub 2010 Aug. 23 (2010). Murine plasma levels of sVCAM-1 and sMAdCAM-1 were measured in a single dose experiment at a range of concentrations over a period of 3 weeks for compound of Formula (I) and ELN 476063. The kinetics of sVCAM-1 and sMAdCAM-1 regulation after a single, subcutaneous dose of the compound of Formula (I) or ELN 476063 was calculated over a period of 21 days.

Naïve C57BL/6 mice were treated with a single, subcutaneous injection of PBS (vehicle), the compound of Formula (I) or ELN 476063 (FIG at a range of doses. Four mice were dosed for each level/timepoint. This was compared against the effects of the non-selective conjugate. Both Formula (I) compound and ELN 476063 caused a rapid, dose-dependent down-regulation of sVCAM-1 and sMAdCAM-1, which recovered as drug levels declined.

The 0.5 mg/kg dose in mice was estimated to be equivalent to the human 0.2 mg/kg dose (based upon Cₘₐₓ). A dose of 0.2 mg/kg in humans would result in a Cₘₐₓ value (0.57 mg/mL) that would be comparable to a Cₘₐₓ (0.83 ng/mL) value obtained in mice at 0.5 mg/kg dose in mice. The 0.5 mg/kg dose in mice was fully efficacious in blocking acute CNS cell trafficking. Using the same analysis, a human dose of 0.5 mg/kg would result in a Cₘₐₓ value of 3.7 μg/mL which would be comparable to a mouse dose of 1 mg/kg (5.5-4.3 ug/mL). A human dose of 0.5 mg/kg is roughly equivalent to a mouse dose of 1.0 mg/kg (by Cₘₐₓ), which was also fully efficacious in preventing acute CNS cell trafficking.

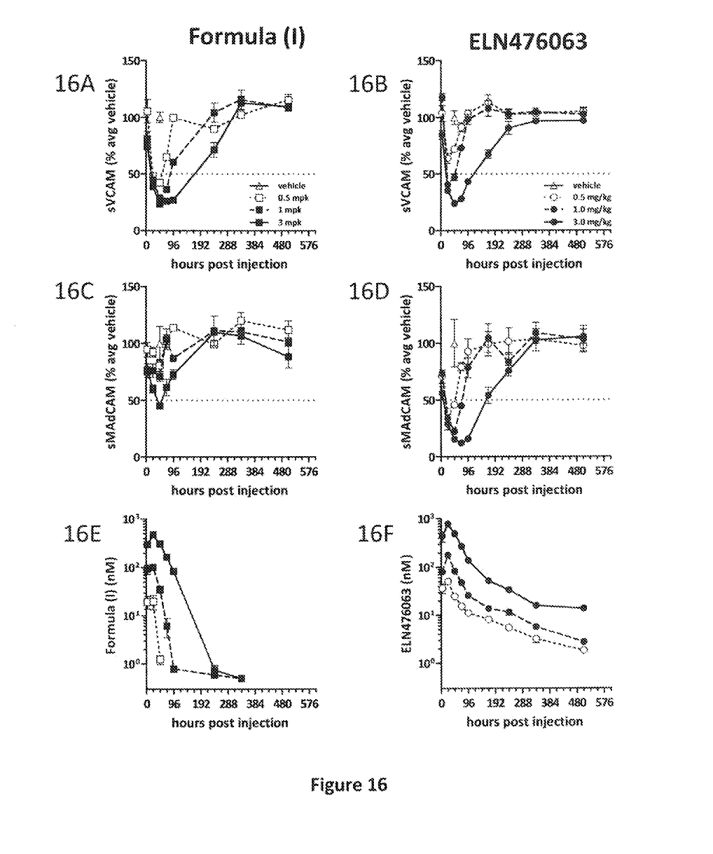

FIGS. 16A-16F show sVCAM-1 (FIGS. 16A and 16B), sMAdCAM-1 (FIGS. 16C and 16D), and plasma drug levels (FIGS. 16 E and F) at various time points ranging from 4 hours to 21 days post-dose. As a point of reference, 50% down-regulation of soluble adhesion molecules based on the average level in vehicle-treated animals is denoted by a dotted line in the graphs of FIG. 16. the rapid, dose-dependent, down-regulation of sVCAM-1 and sMAdCAM-1 resulting from these dosages, which recovered as drug levels decline (see graphs E and F).

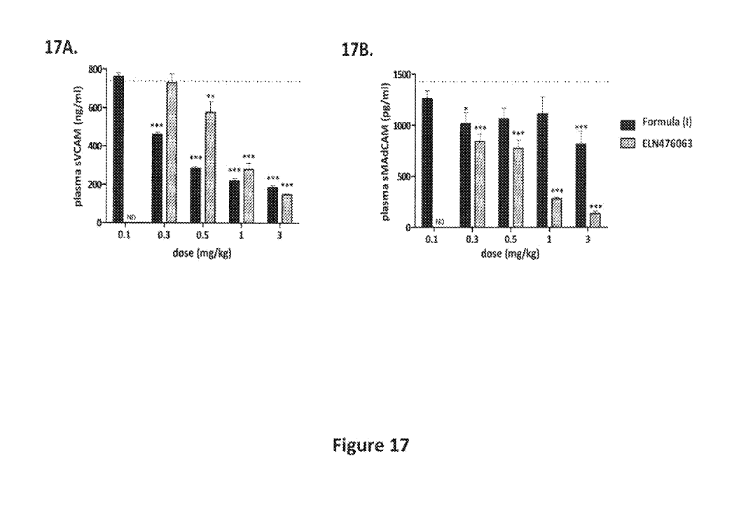

Plasma drug concentrations of Formula (I) compound and ELN 476063 vs sVCAM-1 or sMAdCAM-1 were plotted for each mouse used in the experiment summarized in FIG. 16. Curves were fitted using variable-slope non-linear regression. In agreement with in vitro selectivity data Formula (I) compound more potently down-regulated sVCAM-1 but minimally affected sMAdCAM-1 levels when compared to ELN 476063 Maximal inhibition of sVCAM-1 was seen at Formula (I) compound plasma concentrations as low as 3 nM, whereas sMAdCAM-1 was not fully down-regulated even at the highest plasma concentration (FIG. 17A). In contrast, sVCAM-1 and sMAdCAM-1 are similarly down-regulated with equivalent plasma concentrations of ELN 476063 (FIG. 17B).

The selective down-regulation of the soluble form of the α4β1 ligand VCAM-1 over the α4β7 ligand MAdCAM-1 demonstrate that in vitro selectivity is maintained in vivo through selective regulation of soluble adhesion molecules and points to the in vitro selectivity and potency of the compound of Formula (I) for α4β1 over α4β7 as reflected in vivo via regulation of sVCAM-1 over sMAdCAM-1.

### Example 12

### Selective Inhibition of Cell Trafficking by Administration of Formula (I)

Radiolabeled splenocytes and lymph node cells were adoptively transferred into treated EAE mice and total radioactivity in various organs was quantified. Briefly, experimental autoimmune encephalomyelitis (EAE) was induced in C57BL/6 mice via subcutaneous injection of MOG₃₅₋₅₅ peptide emulsified in CFA followed by intraperitoneal injections of pertussis toxin on days 0 and 2. At day 12 post-induction, mice that had clinical scores of 2 (affected hind-limb gait) were enrolled into the study and dosed subcutaneously with PBS, ELN476063, or Formula (I) at a range of doses. Splenocytes and mesenteric lymph node cells from naïve mice were cultured overnight, labeled in vitro with ¹¹¹Indium, and adoptively transferred into enrolled EAE mice on day 13 post-induction. Approximately 16 hours post-cell transfer, organs were harvested, weighed, and radioactivity was quantified by gamma counter.

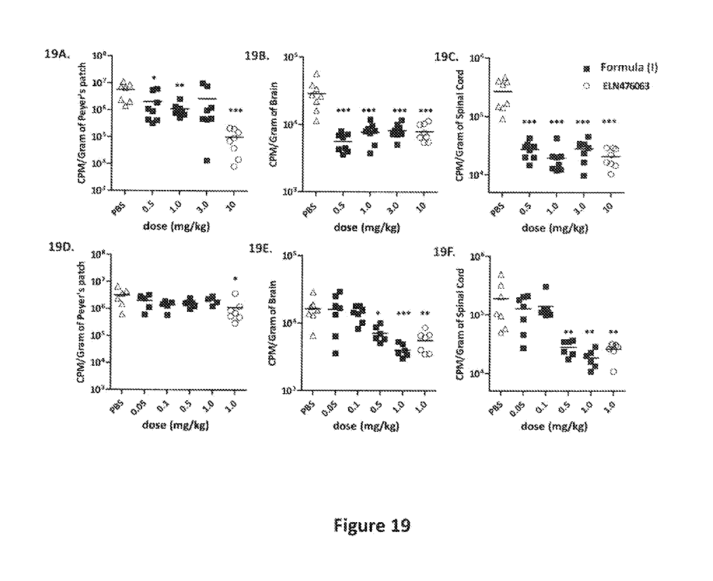

Formula (I) compound was able to significantly inhibit the trafficking of radiolabeled cells into the spinal cords and brains of EAE mice at doses between 0.5 and 3.0 mg/kg, but not at 0.1 mg/kg and below (FIGS. 19B, 19C, 19E and 19F). Formula (I) compound did not greatly affect cell trafficking to the Peyer's patches of the small intestine, which was likely in part mediated by α4β7 interactions (FIGS. 19A and 19D). Inhibition of trafficking to the Peyer's patch and CNS was observed with ELN 476063 at a 10 and 1.0 mg/kg dose.

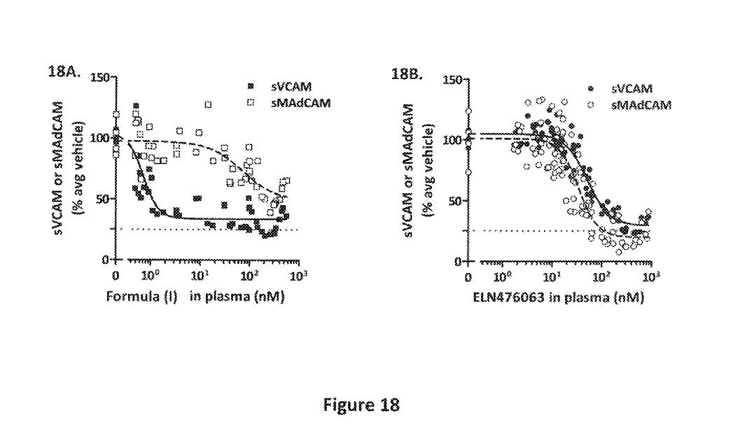

sVCAM-1 is down-regulated at Formula (I) compound doses that block CNS trafficking, but not at 0.1 mg/kg (FIG. 18A), which indicates that sVCAM-1 is a potential marker of not only drug level, but drug activity. Significant inhibition of cell trafficking into the CNS was seen at Formula (I) compound doses that did not greatly affect sMAdCAM-1 levels demonstrating that compound of Formula (I) modulated CNS inflammation without preventing α4β7-dependent immune cell trafficking (FIG. 18B). In fact, as shown in FIG. 15B, levels of sMAdCAM in plasma (which correlate to levels found within the bloodstream) remain at least the 75% normal levels following dosing with Formula (I)—even at doses of up to 3 mg/kg. Thus, administration of Formula (I) does not affect signaling through sMAdCAM at levels shown to have a statistically significant downregulation of sVCAM-1 levels.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims. In the claims that follow, unless the term “means” is used, none of the features or elements recited therein should be construed as means-plus-function limitations pursuant to 35 U.S.C. §112, ¶6.

What is claimed is:

1. A method of treating a disease mediated by α4β1-mediated leukocyte trafficking into the CNS while substantially preserving α4β7-mediated trafficking, comprising administering a compound of Formula (I) to a subject in need of such treatment at a therapeutically effective dose and interval.

2. The method of claim 1, wherein the dose is from about 0.01 mg/kg to about 5 mg/kg.

3. The method of claim 2, wherein the dose is from about 0.2 mg/kg to about 1.0 mg/kg.

4. The method of claim 3, wherein the dose is about 0.2 mg/kg to about 0.5 mg/kg.

5. The method of claim 1, wherein the dose is from about 1.0 mg/kg to about 2.0 mg/kg.

6. The method of claim 2, wherein the compound of Formula (I) is administered weekly.

7. The method of claim 2, wherein the compound of Formula (I) is administered monthly.

8. The method of claim 1, wherein the dose or interval of Formula (I) administration is selected to maintain sMAdCAM levels in the subject's bloodstream at 75% or more following administration compared to sMAdCAM levels in the subject's bloodstream prior to administration of Formula (I).

9. A method of selectively inhibiting α4β1 activity in a subject, comprising administering to the subject a therapeutically effective amount of a conjugate comprising two or more α4β1 small molecule antagonists covalently attached to a biocompatible polymer, wherein the conjugate has a 20 to 100 fold greater potency towards α4β1 than α4β7.

10. The method of claim 9, wherein the conjugate has a 30 to 80 fold greater potency towards α4β1 than α4β7.

11. The method of claim 10, wherein the conjugate comprises the compound of Formula (I).

12. The method of claim 9, wherein the conjugate is administered to a subject with an autoimmune disease.

13. The method of claim 12, wherein the conjugate is administered to a subject with multiple sclerosis.

14. The method of claim 9, wherein the conjugate is administered to a subject with an inflammatory disease.

15. The method of claim 9, wherein the conjugate is administered to a subject with a cell proliferative disorder.

16. The method of claim 9, wherein the conjugate is administered to a subject with to prevent transplant rejection or graft versus host disease.

17. The method of claim 9, wherein the conjugate is administered to a subject to promote neuroprotection following injury.

18. The method of claims 9, wherein the dose or interval of administration of the conjugate is selected to maintain sMAdCAM levels in the subject's bloodstream at 75% or more following administration compared to sMAdCAM levels in the subject's bloodstream prior to administration of Formula (I).

19. A method of treating multiple sclerosis in a subject in need thereof, comprising administering to the subject a compound having a 30 to 80 fold greater potency towards α4β1 than α4β7 in a dose from about 0.01 mg/kg to about 5 mg/kg.

20. The method of claim 19, wherein the dose is from about 0.2 mg/kg to about 1.0 mg/kg.

21. The method of claim 20, wherein the dose is about 0.2 mg/kg to about 0.5 mg/kg.

22. The method of claim 19, wherein the dose is from about 1.0 mg/kg to about 2.0 mg/kg.

23. The method of claim 19, wherein the compound is administered weekly.

24. The method of claim 19, wherein the compound is administered monthly.

25. A method of treating an autoimmune disease in a subject in need thereof, comprising administering to the subject a compound having a 30 to 80 fold greater potency towards α4β1 than α4β7 in a dose from about 0.01 mg/kg to about 5 mg/kg.

26. The method of claim 25, wherein the dose is from about 0.2 mg/kg to about 1.0 mg/kg.

27. The method of claim 26, wherein the dose is about 0.2 mg/kg to about 0.5 mg/kg.

28. The method of claim 25, wherein the dose is from about 1.0 mg/kg to about 2.0 mg/kg.

29. The method of claim 25, wherein the compound is administered weekly.

30. The method of claim 25, wherein the compound is administered monthly.

31. A method of treating an inflammatory disease in a subject in need thereof, comprising administering to the subject a compound having a 30 to 80 fold greater potency towards α4β1 than α4β7 in a dose from about 0.01 mg/kg to about 5 mg/kg.

32. The method of claim 31, wherein the dose is from about 0.2 mg/kg to about 1.0 mg/kg.

33. The method of claim 32, wherein the dose is about 0.2 mg/kg to about 0.5 mg/kg.

34. The method of claim 31, wherein the dose is from about 1.0 mg/kg to about 2.0 mg/kg.

35. The method of claim 31, wherein the compound is administered weekly.

36. The method of claim 31, wherein the compound is administered monthly.

37. A method of treating a cell proliferative disorder in a subject in need thereof, comprising administering to the subject a compound having a 30 to 80 fold greater potency towards α4β1 than α4β7 in a dose from about 0.01 mg/kg to about 10 mg/kg.

38. The method of claim 37, wherein the dose is from about 0.2 mg/kg to about 1.0 mg/kg.

39. The method of claim 38, wherein the dose is about 0.2 mg/kg to about 0.5 mg/kg.

40. The method of claim 37, wherein the dose is from about 1.0 mg/kg to about 2.0 mg/kg.

41. The method of claim 37, wherein the compound is administered monthly.

42. The method of claim 37, wherein the compound is administered monthly.

43. A method of treating an autoimmune disease that is mediated in part by α4β1 integrin receptors while sparing α4β7 mediated immune cell interactions in a patient comprising administering to the patient a weekly, bi-weekly or monthly dose of a compound of Formula (I) of from about 0.2 mg/kg to about 1.0 mg/kg.

44. A method of treating an autoimmune disease that is mediated in part by α4β1 integrin receptors while sparing α4β7 mediated immune cell surveillance in a patient comprising administering to the patient a weekly, bi-weekly or monthly dose of a compound of Formula (I) of from about 0.2 mg/kg to about 1.0 mg/kg.
